SEQUENCE LISTING (5 sequences)

SEQ ID NO: 1 - DNA, 21 bp, synthetic construct
  source 1..21: mol_type = other DNA
ctcagggcggactgggtgctc

SEQ ID NO: 2 - DNA, 39 bp, synthetic construct
  source 1..39: mol_type = other DNA
cagtcccgcctgagaaaaaaaaaaaaaaaaaagagcacc

SEQ ID NO: 3 - DNA, 42 bp, synthetic construct
  source 1..42: mol_type = other DNA
aaacagtcccgcctgagaaaaaaaaaaaaaaaaaagagcacc

SEQ ID NO: 4 - DNA, 42 bp, synthetic construct
  source 1..42: mol_type = other DNA
cagtcccgcctgagaaaaaaaaaaaaaaaaaagagcaccaaa

SEQ ID NO: 5 - DNA, 45 bp, synthetic construct
  source 1..45: mol_type = other DNA
aaacagtcccgcctgagaaaaaaaaaaaaaaaaaagagcaccaaa

(19) United States
(12) Patent Application Publication
(10) Pub. No.: US 2025/0066867 A1
(43) Pub. Date: Feb. 27, 2025

(54) RAPID DETECTION OF KNOWN AND EMERGING INFLUENZA VIRUSES

(71) Applicant: VedaBio, Inc., San Diego, CA (US)

(72) Inventor: Christine Ginocchio, San Diego, CA (US)

(21) Appl. No.: 18/812,721

(22) Filed: Aug. 22, 2024

Related U.S. Application Data
(60) Provisional application No. 63/534,324, filed on Aug. 23, 2023.

(51) Int. Cl. C12Q 1/70; C12Q 1/6818

(52) U.S. Cl. CPC C12Q 1/701; C12Q 1/6818

(57) ABSTRACT

Presented are assay modules for performing multiplex assays that detect many influenza virus-related nucleic acids simultaneously, including those from known influenza viruses and influenza virus subtypes as well as previously unknown, emerging influenza viruses and influenza subtypes, as well as other viruses and bacteria known to cause respiratory symptoms in humans and other animals. Neither sample preparation methods nor the assays necessarily require amplification of the sample nucleic acids yet the assays retain sensitivity, allow for multiplexing, and provide low cost, minimum automated workflow and results in less than thirty minutes.

### RELATED CASES

This application claims priority to U.S. Ser. No. 63/534,324, filed 23 Aug. 2023, which is incorporated by reference in its entirety.

### FIELD OF THE INVENTION

The present disclosure relates to assay modules on which multiplexed nucleic acid assays are performed to detect target nucleic acids from influenza and other respiratory viruses in a sample preferably without amplification of sample nucleic acids.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Rapid and accurate identification—diagnosis—of infectious agents is important in order to select correct treatment and to track the spread of epidemics. This is particularly true regarding influenza viruses. In a typical year, 5-15% of the population contracts influenza, with 3-5 million severe cases and up to 650,000 respiratory-related deaths. Moreover, there have been large outbreaks of novel influenza strains that spread globally (i.e., pandemics) that have occurred every 10-50 years, including the Spanish flu pandemic of 1918-1920, which was the most severe flu pandemic known. The World Health Organization (WHO) and the US Centers for Disease Control (CDC) have expressed great concern that a novel highly pathogenic avian or swine influenza virus will mutate and rapidly become human-to-human transmissible, leading to another global pandemic. In the design of the present assays and assay modules, the Influenza Risk Assessment Tool (IRAT) was taken into consideration. The IRAT is an evaluation tool developed by CDC and external influenza experts that assesses the potential pandemic risk posed by influenza A viruses that currently circulate in animals but not in humans. The IRAT assesses potential pandemic risk based on two different questions regarding “emergence” and “public health impact.” Rapid identification of a new avian/swine or human influenza strain is critical for protecting both animal and human populations.

Needed in the art are diagnostic technologies that facilitate sample splitting and reagent partitioning to empower very rapid and accurate identification of emerging infectious strains of influenza and other respiratory viruses in a single test. The present disclosure satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides assay modules for performing multiplex assays that detect many influenza virus-related nucleic acids simultaneously, including those from known influenza viruses and influenza virus subtypes as well as previously unknown, emerging influenza viruses and influenza subtypes. The key to the present disclosure is that a sample is split and delivered to a plurality of partitions, allowing for identification of influenza A (via one or more nucleic acids complementary to matrix proteins); influenza B (via one or more nucleic acids complementary to hemagglutinin proteins and/or nucleoproteins); influenza A hemagglutinin subtype nucleic acids of interest, such as H1, H3 and H5 (as well as H2, H4, H6, H7, H8, H9, H10, and/or emergent hemagglutinin types of concern); influenza A specific lineages, neuraminidase subtype nucleic acids of interest (such as N1 and N2); as well as nucleic acids complementary to other respiratory viruses and/or bacteria of interest. Although specific embodiments described herein involve signal boost assays that do not require amplification of sample nucleic acids, assays that do utilize amplification may also be employed.

In some embodiments there is provided an assay module for identifying one or more influenza virus-related nucleic acids in a sample comprising: a plurality of partitions comprising reagents to identify one or more influenza virus-related nucleic acids, where the following separate partitions are present: a partition to identify an influenza A virus-related nucleic acids; a partition to identify one or more influenza B virus-related nucleic acids; a partition to identify one or more influenza A virus H1 hemagglutinin subtype nucleic acids; a partition to identify one or more influenza A virus H3 hemagglutinin subtype nucleic acids; and partitions to identify one or more influenza A virus neuraminidase subtype nucleic acids, such as N1 and N2 or others of interest.

In some aspects of this embodiment, the reagents to identify the influenza A virus nucleic acid identify an influenza A matrix protein nucleic acid, and in some aspects the reagents to identify the influenza B virus identify influenza B hemagglutinin protein or nucleoprotein nucleic acids. In some aspects, the assay module further comprises a partition comprising reagents to identify any relevant anti-viral resistance mutations—for example, the H275Y mutation leading to oseltamivir resistance in the influenza A virus neuraminidase nucleic acids of H1N1pdm09 virus—and in some aspects, the assay module further comprises one or more partitions comprising reagents for a control.

In some aspects, the assay module further comprises one or more partitions comprising reagents to identify one or more of additional influenza A hemagglutinin subtype nucleic acids such as H2, H4, H5, H6, H7, H8, H9, H10 hemagglutinin subtype, emergent hemagglutinin types of concern, specific lineages of influenza and/or one or more of an influenza A virus neuraminidase subtype such as N3, N7, N8 or N9 neuraminidase subtype.

In some aspects, the assay module further comprises one or more partitions comprising reagents to one or more nucleic acids selected from seasonal coronaviruses (e.g., OC43, NL63, HKU-1, 229E); severe acute respiratory syndrome coronavirus 2 (SARS CoV-2); adenovirus (AdV), respiratory syncytial virus (RSV); parainfluenza viruses (PIV) 1,2,3, and/or 4; human metapneumovirus (HMPV); rhinovirus/enterovirus (R/EV); and/or one or more nucleic acids selected from Bordetella parapertussis, Bordetella pertussis, Chlamydia pneumoniae, and/or Mycoplasma pneumoniae, or pools of such nucleic acids.

In a specific example of an assay module as described below, the present disclosure provides assay modules upon which multiplexed assay methods are performed to detect influenza virus nucleic acids in a sample without the need for amplification of the influenza virus nucleic acids although amplification optionally may be used in some embodiments. The assay allows for detection of both known and unknown (e.g., new variant) influenza viruses. The “signal boost assays” or “signal boost nuclease assays” described herein comprise two different ribonucleoprotein (RNP) complexes and blocked split activator molecules, which allow for multiplexing without sacrificing sensitivity.

In the signal boost assay embodiments described herein, blocked split activator molecules in the signal boost assay keep second ribonucleoprotein complexes (RNP2s) “locked” unless and until an influenza virus nucleic acid activates a first ribonucleoprotein complex (RNP1). The present signal boost assays can detect influenza virus nucleic acids at femtomolar (fM) limits without the need for amplifying the nucleic acid(s) in the sample, thereby avoiding the drawbacks of multiplex RNA/DNA amplification, such as primer-dimerization. A particularly advantageous feature of the signal boost assay is that, with the exception of the gRNAs in RNP1, the signal boost assay components may be the same in each assay no matter what target nucleic acids of interest are being detected; moreover, the gRNAs in the RNP1 are easily reprogrammed using traditional guide design methods meaning that the signal boost assay can be changed quickly as needed to detect new emerging strains of influenza or other respiratory viruses. (For more information on the signal boost assays, including additional embodiments, see, e.g., see U.S. Pat. Nos. 11,693,520; 11,702,686; 11,821,025; 11,970,730; 11,987,839; 11,884,921; 11,820,983; 11,859,182; 11,884,922; and 11,946,052.)

In some embodiments of the assay module, the assay module further comprises: a sample splitting region comprising at least one inlet and fluid channels fluidically coupled to the separate partitions, where each partition comprises a plurality of first ribonucleoprotein complexes (RNP1s), where the RNP1s comprise a first nucleic acid nuclease and a first guide nucleic acid (gRNA1), and where a first partition has one or more RNP1s with one or many different gRNA1s complementary to the influenza A virus-related nucleic acid; a second partition has one or more RNP1s with one or many different gRNA1s complementary to the influenza B virus-related nucleic acid; a third partition has one or more RNP1s with one or many different gRNA1s complementary to the influenza A virus H1 hemagglutinin subtype nucleic acid; a fourth partition has one or more RNP1s with one or many different gRNA1s complementary to the influenza A virus H2 hemagglutinin subtype nucleic acid; a fifth partition has one or more RNP1s with one or many different gRNA1s complementary to the influenza A virus H3 hemagglutinin subtype nucleic acid; a sixth partition has one or more RNP1s with one or many different gRNA1s complementary to an influenza A virus neuraminidase subtype nucleic acid; and a seventh partition has one or more RNP1s with one or many different gRNA1s complementary to a second influenza A virus neuraminidase subtype nucleic acid.

Thus, in some aspects, the reagents to identify the influenza A virus nucleic acid, influenza B virus nucleic acid, influenza A hemagglutinin subtype nucleic acids and influenza A neuraminidase subtype nucleic acids are ribonucleoprotein complexes. In some additional embodiments there may be pooled RNP1s within a single partition for, e.g., testing variants in the seasonal coronaviruses (CoVs) (OC43, NL63, HKU-1, 229E), SARS CoV-2, AdV, RSV, PIV 1,2,3,4, HMPV, and R/EV, as well as for testing bacterial pathogens. That is, a single partition may comprise different RNP1s that detect different variants or subvariants of, e.g., SARS-CoV-2. Moreover, there may be pools of RNP1s in a single partition that detect two or more of, e.g., seasonal coronaviruses (CoVs) (OC43, NL63, HKU-1, 229E), SARS CoV-2, AdV, RSV, PIV 1,2,3,4, HMPV, and R/EV; or pools of RNP1s in a single partition that detect two or more of, e.g., bacterial pathogens.

In some aspects of these embodiments, the assay module further comprises: the RNP2s; the blocked split activator molecules; and the reporter moieties in each of the partitions comprising the RNP1s; however, as described below, many different configurations are possible.

The assay modules may further comprise a pump configured to provide negative or positive pressure to the sample splitting region; and a detection and imaging zone. The assay modules may further comprise valves between the sample splitting region and the fluid channels fluidically coupled to the first partitions.

In some aspects, one or more of the RNP1s, RNP2s, blocked split activator molecules and reporter moieties are lyophilized and in some aspects, one or more of the RNP1s, RNP2s, blocked split activator molecules and reporter moieties are air dried.

These aspects and other features and advantages of the invention are described below in more detail.

### BRIEF DESCRIPTION OF THE DRAWINGS

The foregoing and other features and advantages of the present invention will be more fully understood from the following detailed description of exemplary embodiments taken in conjunction with the accompanying drawings in which:

FIG. 1A is a schematic overview of an exemplary influenza identification assay module.

FIG. 1B is a schematic overview of three exemplary embodiments of methods for performing the signal boost assay on an assay module.

FIG. 1C shows embodiments of two assay modules.

FIG. 2A is an overview of the general principles underlying the nucleic acid-guided nuclease signal boost assay involving two different types of ribonucleoprotein complexes (RNPs) as described in detail herein.

FIG. 2B is a diagram showing the sequence of steps in an signal boost assay utilizing blocked nucleic acid molecules.

FIG. 3 is a simplified diagram of an exemplary gRNA2 sequence and three exemplary blocked split activator molecules.

FIG. 4 is a simplified diagram showing the sequence of steps in an exemplary cascade assay utilizing blocked split activator molecules.

FIG. 5A is a graphic illustration of an exemplary workflow for an assay module.

FIG. 5B is an illustration of an exemplary sample splitting assay module.

It should be understood that the drawings are not necessarily to scale, and that like reference numbers refer to like features.

### DEFINITIONS

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

All of the functionalities described in connection with one embodiment of the compositions and/or methods described herein are intended to be applicable to the additional embodiments of the compositions and/or methods except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Note that as used herein and in the appended claims, the singular forms “a,” “an,” and “the” include plural referents unless the context clearly dictates otherwise. Thus, for example, reference to “a cell” refers to one or more cells, and reference to “a system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA, as RNA, or a combination of DNA and RNA (e.g., a chimeric nucleic acid).

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both limits, ranges excluding either or both of those included limits are also included in the invention.

The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context.

As used herein, the terms “binding affinity” or “dissociation constant” or “K_{d}” refer to the tendency of a molecule to bind (covalently or non-covalently) to a different molecule. A high K_{d} (which in the context of the present disclosure refers to blocked split activator molecules binding to RNP2) indicates the presence of more unbound molecules, and a low K_{d} (which in the context of the present disclosure refers to unblocked split activator molecules binding to RNP2) indicates the presence of more bound molecules. In the context of the present disclosure and the binding of blocked or unblocked split activator molecules to RNP2, low K_{d} values are in a range from about 100 fM to about 1 aM or lower (e.g., 100 zM) and high K_{d} values are in the range of 100 nM-100 μM (10 mM) and thus are about 10⁵- to 10¹⁰-fold or higher as compared to low K_{d} values.

As used herein, the terms “binding domain” or “binding site” refer to a region on a protein, DNA, or RNA to which specific molecules and/or ions (ligands) may form a covalent or non-covalent bond. By way of example, a polynucleotide sequence present on a nucleic acid molecule may serve as a binding domain for a different nucleic acid molecule. Characteristics of binding sites are chemical specificity, a measure of the types of ligands that will bond, and affinity, which is a measure of the strength of the chemical bond.

As used herein, the terms “blocked nucleic acid molecule” or “high K_{d} nucleic acid” refer to nucleic acid molecules that cannot bind to the first or second RNP complex to activate cis- or trans-cleavage (or bind with very low affinity). “Unblocked nucleic acid molecule” refers to a formerly blocked nucleic acid molecule that can bind to the second RNP complex (RNP2) to activate trans-cleavage of additional blocked nucleic acid molecules.

As used herein, the term “blocked split activator molecule” refers to nucleic acid molecules that cannot bind to the first or second ribonucleoprotein complex (RNP) (i.e., RNP1 or RNP2) of the cascade assay to activate cis-or trans-cleavage. “Unblocked split activator molecule” refers to a formerly blocked split activator molecule that can bind to the second RNP complex (RNP2) to activate trans-cleavage of additional blocked split activator molecules; that is, an “unblocked split activator molecule” is the target nucleic acid for RNP2.

The terms “Cas RNA-guided nucleic acid-guided nuclease” or “CRISPR nuclease” or “nucleic acid-guided nuclease” refer to a CRISPR-associated protein that is an RNA-guided nucleic acid-guided nuclease suitable for assembly with a sequence-specific gRNA to form a ribonucleoprotein (RNP) complex.

As used herein, the terms “cis-cleavage”, “cis-nucleic acid-guided nuclease activity”, “cis-mediated nucleic acid-guided nuclease activity”, “cis-nuclease activity”, “cis-mediated nuclease activity”, and variations thereof refer to sequence-specific cleavage of a target nucleic acid of interest (here, an influenza virus nucleic acids), including an unblocked split activator molecule by a nucleic acid-guided nuclease in an RNP complex. Cis-cleavage is a single turn-over cleavage event in that only one substrate molecule is cleaved per event.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-ATCGAT-5′ is 100% complementary to a region of the nucleotide sequence 5′-GCTAGCTAG-3′.

As used herein, the term “contacting” refers to placement of two moieties in direct physical association, including in solid or liquid form. Contacting can occur in vitro with isolated cells (for example in a tissue culture dish or other vessel) or in samples or in vivo by administering an agent to a subject.

A “control” is a reference standard of a known value or range of values.

The terms “guide nucleic acid” or “guide RNA” or “gRNA” refer to a polynucleotide comprising 1) a crRNA region or guide sequence capable of hybridizing to the target strand of a target nucleic acid of interest, and 2) a scaffold sequence capable of interacting or complexing with a nucleic acid-guided nuclease. The crRNA region of the gRNA is a customizable component that enables specificity in every nucleic acid-guided nuclease reaction. A gRNA can include any polynucleotide sequence having sufficient complementarity with a target nucleic acid of interest to hybridize with the target nucleic acid of interest and to direct sequence-specific binding of a ribonucleoprotein (RNP) complex containing the gRNA and nucleic acid-guided nuclease to the target nucleic acid.

“Modified” refers to a changed state or structure of a molecule. Molecules may be modified in many ways including chemically, structurally, and functionally. In one embodiment, a nucleic acid molecule (for example, a blocked split activator molecule) may be modified by the introduction of non-natural nucleosides, nucleotides, and/or internucleoside linkages. In another embodiment, a modified protein (e.g., a modified or variant nucleic acid-guided nuclease) may refer to any polypeptide sequence alteration which is different from the wildtype.

As used herein, a “partition” is an isolate region (e.g., a feature surrounded by an interstitial region) or an isolate depression (e.g., a well) on a substrate, or a droplet. Partitions are used, in relation to the present disclosure, to compartmentalize a plurality of ribonucleoprotein complexes (RNP1s) comprising different guide nucleic acids (gRNA1s) and/or other assay components into, e.g., separate wells, features, or droplets.

The terms “percent sequence identity”, “percent identity”, or “sequence identity” refer to percent (%) sequence identity with respect to a reference polynucleotide or polypeptide sequence following alignment by standard techniques. Alignment for purposes of determining percent sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5):1792-1797 (2004)). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul, et al., J. Mol. Biol., 215:403-410 (1990)).

As used herein, the terms “preassembled ribonucleoprotein complex”, “ribonucleoprotein complex”, “RNP complex”, or “RNP” refer to a complex containing a guide RNA (gRNA) and a nucleic acid-guided nuclease, where the gRNA is complexed with (i.e., non-covalently coupled with) the nucleic acid-guided nuclease. The gRNA (i.e., here the gRNA in the first ribonucleoprotein complex (gRNA1 in RNP1)), which includes a sequence complementary to a target nucleic acid of interest (here, an influenza virus nucleic acid), guides the RNP to the target nucleic acid of interest and hybridizes to it. The hybridized target nucleic acid/gRNA units are cleaved by the nucleic acid-guided nuclease. In the signal boost assays described herein, a first ribonucleoprotein complex (RNP1) includes a first guide RNA (gRNA) specific to a target nucleic acid of interest, and a first nucleic acid-guided nuclease, such as, for example, Cas12a or Cas14a for a DNA target nucleic acid or Cas13a or Cas12g for an RNA target nucleic acid. A second ribonucleoprotein complex (RNP2) used for boosting the signal from reporter moieties includes a second guide RNA (gRNA2) specific to an unblocked nucleic acid molecule or unblocked split activator molecule and a second nucleic acid-guided nuclease, which may be different from or the same as the first nucleic acid-guided nuclease, again, Cas12a or Cas14a for a DNA unblocked nucleic acid molecule or unblocked split activator molecule or Cas13a or Cas12g for an RNA unblocked nucleic acid molecule or unblocked split activator molecule.

As used herein, the terms “protein” and “polypeptide” are used interchangeably. Proteins may or may not be made up entirely of amino acids.

As used herein, the term “sample” refers to body fluids, including but not limited to broncheoalveolar lavage fluid, lavage fluids from sinus cavities, lavage fluids from nasal passages, nasal swabs, nasopharyngeal swabs, endotracheal aspirates or bronchopulmonary aspirates.

The terms “target nucleic acid of interest”, “target sequence”, “target nucleic acid molecule of interest”, “target molecule of interest”, “target nucleic acid”, or “target of interest” refer to any locus that is recognized by a gRNA sequence in vitro or in vivo. In the present disclosure, the target nucleic acids of interest are “influenza virus nucleic acids of interest” or “influenza virus nucleic acids.” The “target strand” of a target nucleic acid of interest is the strand of the double-stranded target nucleic acid that is complementary to a gRNA. The spacer sequence of a gRNA may be 50%, 60%, 75%, 80%, 85%, 90%, 95%, 97.5%, 98%, 99% or more complementary to the target nucleic acid of interest. Optimal alignment can be determined with the use of any suitable algorithm for aligning sequences. Full complementarity is not necessarily required provided there is sufficient complementarity to cause hybridization and trans-cleavage activation of an RNP complex. The target nucleic acid of interest, if a DNA target nucleic acid of interest, may be associated with a protospacer adjacent motif (PAM) sequence, which may include a 2-5 base pair sequence adjacent to the protospacer. In some embodiments 1, 2, 3, 4, 5, 6, 7, 8, 9, 10, or more target nucleic acids can be detected by the disclosed method.

As used herein, the terms “trans-cleavage”, “trans-nucleic acid-guided nuclease activity”, “trans-mediated nucleic acid-guided nuclease activity”, “trans-nuclease activity”, “trans-mediated nuclease activity” and variations thereof refer to indiscriminate, non-sequence-specific cleavage of a target nucleic acid molecule by a nucleic acid-guided nuclease (such as by a Cas12, Cas13, and Cas14) which is triggered by binding of N nucleotides of a target nucleic acid molecule (here, an influenza virus nucleic acid, another viral or bacterial nucleic acid or an unblocked split activator molecule) to a gRNA. Trans-cleavage is a “multiple turn-over” event, in that more than one substrate molecule is cleaved after initiation by a single turn-over cis-cleavage event.

Type V CRISPR/Cas nucleic acid-guided nucleases are a subtype of Class 2 CRISPR/Cas effector nucleases such as, but not limited to, engineered Cas12a, Cas12b, Cas12c, Cas12g C2c4, C2c8, C2c5, C2c10, C2c9, CasX (Cas12e), CasY (Cas12d), Cas 13a nucleases or naturally-occurring proteins, such as a Cas12a isolated from, for example, Francisella tularensis subsp. novicida (Gene ID: 60806594), Candidatus methanoplasma termitum (Gene ID: 24818655), Candidatus methanomethylophilus alvus (Gene ID: 15139718), and [Eubacterium] eligens ATCC 27750 (Gene ID: 41356122), and an artificial polypeptide, such as a chimeric protein.

### DETAILED DESCRIPTION

The present disclosure provides assay modules for performing multiplex assays that detect many influenza virus-related nucleic acids simultaneously, including those from known influenza viruses and influenza virus subtypes as well as previously unknown, emerging influenza viruses and influenza subtypes. Neither the sample preparation methods nor the assays require amplification of the influenza virus nucleic acids yet the assays retain sensitivity although amplification may be employed in certain embodiments if desired. Further, the assays allow for multiplexing, involve minimum automated workflow, are low cost and provide results in less than ten minutes.

Rapid and accurate identification—diagnosis—of infectious agents is critical in order to select correct treatment and to track the spread of epidemics. This is particularly true regarding influenza viruses. In a typical year, 5-15% of the population contracts influenza, with 3-5 million severe cases and up to 650,000 respiratory-related deaths. Moreover, there have been pandemics that have occurred every 10-50 years, including five flu pandemics that have occurred since 1900: the Spanish flu pandemic in 1918-1920, which was the most severe flu pandemic; the Asian flu pandemic in 1957; the Hong Kong flu pandemic in 1968; the Russian flu pandemic in 1977; and the swine flu pandemic in 2009. The World Health Organization (WHO) and the US Centers for Disease Control (CDC) have expressed great concern that a novel highly pathogenic avian or swine influenza virus will mutate and rapidly become human to human transmissible, leading to another global pandemic. On Apr. 1, 2024, the US CDC confirmed one human highly pathogenic avian influenza A (HPAI A [H5N1]) infection in a person with exposure to dairy cows in Texas that was presumed to be infected with the virus. This is thought to be the first instance of likely mammal-to human spread of HPAI A (H5N1) virus. In May 2024, CC began reporting additional sporadic human cases in people who had exposure to infected dairy cows. The Influenza Risk Assessment Tool (IRAT)—an evaluation tool developed by CDC and external influenza experts that assesses the potential pandemic risk posed by influenza A viruses that currently circulate in animals but not in humans (htps://www.cdc.gov/flu/pandemic-respurces/national-strategy/risk-assessment.htm)—is considered in the design of the present assays and assay modules. The IRAT assesses potential pandemic risk based on two different questions regarding “emergence” and “public health impact.” Rapid identification of a new avian/swine or human strain is critical for protecting both animal and human populations.

Influenza, commonly known as “the flu”, is an infectious disease caused by influenza viruses. Symptoms range from mild to severe, often including symptoms such as fever, runny nose, sore throat, muscle pain, headache, coughing and fatigue. Symptoms typically begin from one to four days after exposure to the virus and last for about two to eight days. Diarrhea and vomiting also can occur, particularly in children. Additionally, influenza may progress to pneumonia, which can be caused by the influenza or another virus or by a subsequent bacterial infection. Other complications of infection include acute respiratory distress syndrome, meningitis, encephalitis, and worsening of pre-existing health problems such as asthma and cardiovascular disease.

There are four types of influenza virus: A, B, C, and D. Aquatic birds are the primary source of influenza A virus, which is also widespread in various mammals, including humans and pigs. Influenza B virus and influenza C virus primarily infect humans, and influenza D virus is found in cattle and pigs. Influenza A virus and influenza B virus circulate in humans and cause seasonal epidemics. Influenza C virus causes a mild infection, primarily in children. Influenza D virus can infect humans but is not known to cause illness. In humans, influenza viruses are primarily transmitted through respiratory droplets produced from coughing and sneezing, although transmission through aerosols and intermediate objects and surfaces contaminated by the virus also occur.

Influenza A virus is responsible for most cases of severe illness as well as seasonal epidemics and occasional pandemics. Influenza A virus infects people of all ages but tends to disproportionately cause severe illness in the elderly, the very young, and those who have chronic health issues. Birds are the primary reservoir of influenza A virus, especially aquatic birds such as ducks, geese, shorebirds, and gulls, but the virus also circulates among mammals, including pigs, horses, and marine mammals. Influenza A virus is classified into subtypes based on the viral proteins haemagglutinin (H) and neuraminidase (N). Currently, 18 H subtypes and 11 N subtypes have been identified. Most potential combinations have been reported in birds, but H17-18 and N10-11 have only been found in bats and only H subtypes H1-3 and N subtypes N1-2 are known to have circulated in humans. Common influenza A virus subtypes in circulation are H1N1pdm09 and H3N2.

Influenza B virus mainly infects humans but has been identified in seals, horses, dogs, and pigs. Influenza B virus does not have subtypes like influenza A virus. Until recently two antigenically distinct lineages, termed (B/)Victoria(-like) and (B/)Yamagata (-like) were co-circulating but today only Victoria lineage has been detected. Influenza B disproportionately affects children. Influenza B virus contributes to seasonal epidemics alongside influenza A virus but have never been associated with a pandemic.

Influenza viruses have a negative-sense, single-stranded RNA genome that is segmented. Negative sense means the genome can be used as a template to synthesize messenger RNA (mRNA). Influenza A virus and influenza B virus have eight genome segments that encode 10 major proteins, including a matrix protein (M) for influenza A or nucleoprotein for influenza B as well as the hemagglutinin (H) and neuraminidase (N) proteins. The nucleic acids coding for these proteins can be used to distinguish influenza A from influenza B, as well as to identify influenza A subtypes. It is the mixing of the various gene segments in pigs or waterfowl that leads to genetic shifts and the emergence of new influenza A strains.

The key to the present disclosure is that a sample is split and delivered to a plurality of partitions, allowing for identification of influenza A (via one or more nucleic acids complementary to matrix proteins); influenza B (via one or more nucleic acids complementary to hemagglutinin proteins and/or nucleoproteins); influenza A hemagglutinin subtype nucleic acids of interest, such a H1, H3 and H5 (and H2, H4, H6, H7, H8, H9, H10); influenza A neuraminidase subtype nucleic acids of interest (N3, N7, N8 and/or N9), emerging variants of concern or specific lineages; as well as other respiratory viruses and/or bacteria of interest. Although specific embodiments described herein involve signal boost assays that do not require amplification of sample nucleic acids, assays that do utilize amplification may be employed.

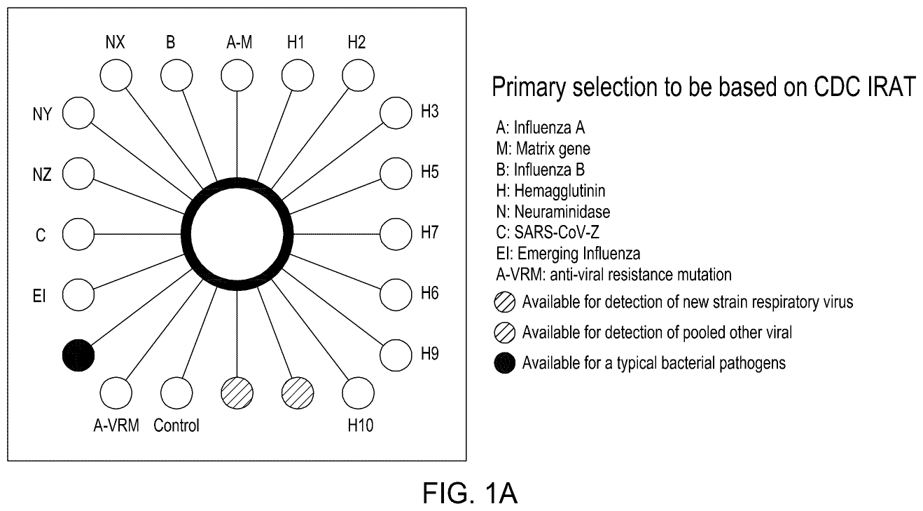

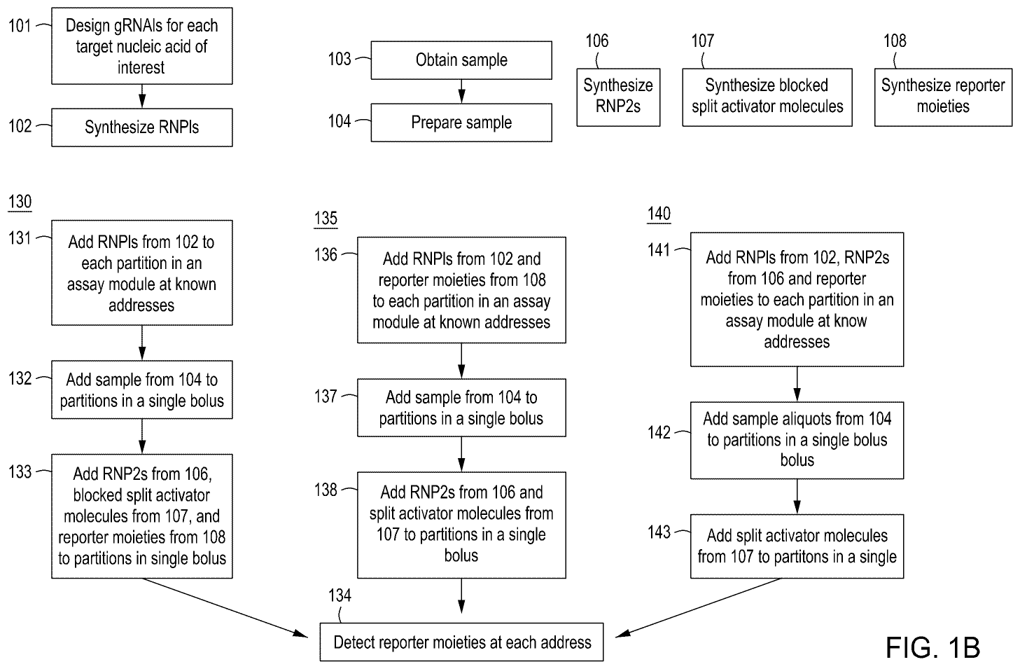

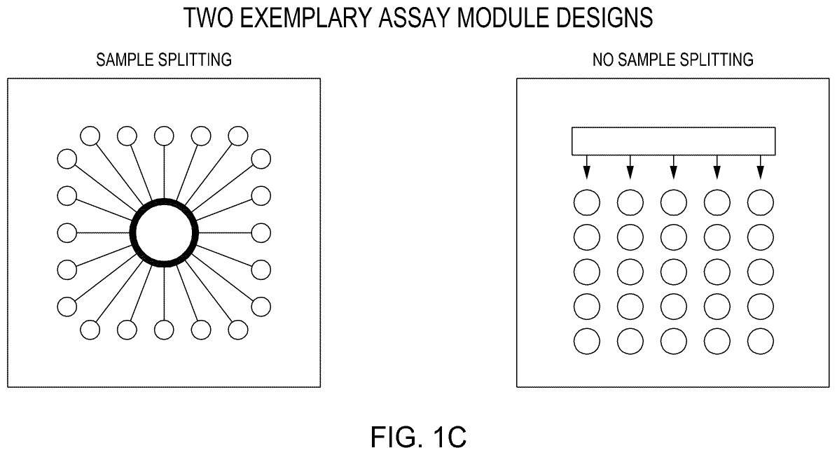

FIG. 1A is an exemplary schematic overview of an influenza identification assay module, which facilitates identification of new and emerging influenza viruses It is envisioned that such a panel could be run alone or as a follow-up to a general respiratory panel at facilities such as state or national public health laboratories, e.g., the Center for Disease Control (CDC) and other global public health laboratories (European Centre for Disease Prevention and Control, World Health Organization), point-of-care facilities, pharmacies, academic centers, as well as locales requiring animal surveillance including farms, auctions, fairs and processing facilities. Comprehensive syndromic testing panels are generally done only in a hospital setting, and testing typically is limited to admitted “sicker” patients due to test cost and restrictions on reimbursement. Limited testing can lead to delays in detection by weeks and a subsequent lack of identifying an “emerging” infectious agent.

FIG. 1A presents an exemplary schematic overview of an influenza identification assay module. Whereas most if not all diagnostic devices for influenza viruses target known strains of influenza A virus (H1N1, HIN2, H3N2, etc.) in addition to influenza B, the present influenza identification module identifies Influenza B as well as two or more of the viral protein haemagglutinin subtypes (H) (e.g., H1, H2, H3, H5, H6, H7, H9 and H10) and optionally one or more of the neuraminidase subtypes (N) (e.g., N1, N2, N3, N7, N8, and N9) separately. Identifying the H subtypes and N subtypes separately provides accurate identification of influenza A subtype combinations, including those previously unknown in humans.

The exemplary assay module shown FIG. 1A provides 20 partitions for identifying respiratory virus and bacterial nucleic acids, including a partition for identifying the influenza A matrix nucleic acid; a partition for identifying the influenza B hemagglutinin or nucleoprotein nucleic acid; a partition for identifying the H1 hemagglutinin subtype; a partition for identifying the H2 hemagglutinin subtype; a partition for identifying the H3 hemagglutinin subtype; a partition for identifying the H5 hemagglutinin subtype; a partition for identifying the H7 hemagglutinin subtype; a partition for identifying the H6 hemagglutinin subtype; a partition for identifying the H9 hemagglutinin subtype; a partition for identifying the H10 hemagglutinin subtype; a partition for identifying one neuraminidase subtype (here, designated NX); a partition for identifying a second neuraminidase subtype (designated NY); a partition for identifying a third neuraminidase subtype (designated NZ); a partition for identifying SARS-CoV-2; and a partition for identifying an emerging influenza.

The influenza A matrix proteins and influenza B hemagglutinin proteins and/or nucleoproteins (and the nucleic acids that code therefor) are generally conserved, with less sequence variation between strains than what is found in the hemagglutinin and neuraminidase genes. A positive result for the presence of, e.g., an influenza A matrix protein, allows for a positive diagnosis for influenza A. If, for example, there is also a positive result for the H1 hemagglutinin subtype and the N1 neuraminidase subtype, a positive diagnosis for influenza A H1/N1 can be made. If, however, there is a positive result for the influenza A matrix protein and the N2 neuraminidase subtype—but no result for a hemagglutinin subtype—it is possible that the influenza A virus in the sample is a variant that has not been previously identified, comprises an unknown hemagglutinin subtype, and should be flagged as a possible emerging influenza A variant. Similarly, if there is a positive result for the influenza A matrix protein and the, e.g., H1 hemagglutinin subtype and no result for a neuraminidase subtype—or there is no result for either the hemagglutinin subtype or neuraminidase subtype—the results should be flagged and re-testing may be conducted or the sample may need to be sent to a government public health testing site for further characterization.

The exemplary assay module in FIG. 1A also includes a partition for identifying anti-viral resistance mutations (in one example, the H275Y mutation in the influenza A N1 gene, which confers resistance to oseltamivir used in the treatment of influenza A and B infections), as well as a partition for a, e.g., positive or negative control. In addition, the assay module in FIG. 1A provides three partitions that can be used for, e.g., nucleic acids that identify other prevalent respiratory viruses such as SARS-CoV-2; pooled nucleic acids that identify other respiratory viruses such as seasonal coronaviruses (CoVs) (OC43, NL63, HKU-1, 229E), AdV, RSV, PIV 1,2,3,4, HMPV, and R/EV; and one or more nucleic acids from atypical bacterial pathogens causing similar symptoms as influenza (B. parapertussis, B. pertussis, C. pneumoniae, and/or M. pneumoniae). That is, it should be apparent to one of ordinary skill in the art given the present disclosure and the description of assay modules below that there may be more than twenty partitions and that in addition to the separate partitions for the hemagglutinin and neuraminidase nucleic acids, other respiratory-related nucleic acids may be included as well. Further, the influenza identification assay module may be used after a general respiratory panel configured to detect, e.g., AdV, influenza A, influenza B, CoVs, SARS-CoV-2, RSV, PIVs, HMPV, R/EV, B. parapertussis, B. pertussis, C. pneumoniae, and/or M. pneumoniae, if the general respiratory panel gives positive results for influenza A. Moreover, there may and typically will be pooled reagents within a single partition for, e.g., testing two or more variants in the neuraminidase and hemagglutinin subtypes; for testing variants and subvariants in the seasonal coronaviruses (CoVs) (OC43, NL63, HKU-1, 229E); or testing other known respiratory viruses such as SARS CoV-2, AdV, RSV, PIV 1,2,3,4, HMPV, and R/EV; and for testing variants in the bacterial pathogens. That is, a single partition may comprise reagents (in the context of the present signal boost assay, many different RNP1s) that detect different variants or subvariants of, e.g., neuraminidase and hemagglutinin nucleic acids, SARS-CoV-2, AdV, etc.

Molecular biology techniques, such as polymerase chain reaction (PCR) and use of nucleic acid-guided nucleases, can be utilized for the detection of influenza virus nucleic acids and may be used in the present assay; however, currently available nucleic acid detection methods such as quantitative PCR (also known as real time PCR or qPCR) or CRISPR-based detection assays such as SHERLOCK™ and DETECTR™ rely on nucleic acid amplification, which requires time and may lead to changes in the relative proportion of nucleic acids, particularly in multiplexed nucleic acid assays. The lack of rapidity for these detection assays is due to the fact that there is a significant lag phase early in the amplification process where fluorescence above background cannot be detected. With qPCR, for example, there is a lag until the cycle threshold or Ct value, which is the number of amplification cycles required for the fluorescent signal to exceed the background level of fluorescence, is achieved and can be quantified. Further, qPCR requires complex instrumentation for thermocycling.

The specific assays of the present disclosure utilize assay modules for performing a signal boost assay that can detect many multiplexed influenza virus nucleic acids in a multiplexed manner at femtomolar (fM) (or lower) limits without the need for amplifying the target nucleic acid(s) of interest, thereby avoiding the drawbacks of multiplex amplification, such as primer-dimerization. The signal boost assays utilize signal boost mechanisms comprising various components including nucleic acid-guided nucleases; guide RNAs (gRNAs) incorporated into ribonucleoprotein complexes (RNPs); blocked split activator molecules, and reporter moieties. A particularly advantageous feature of the signal boost assay is that, with the exception of the gRNA(s) in the RNP1(s) (i.e., gRNA1s), the signal boost assay components may be identical no matter what target nucleic acids of interest are being detected, and gRNA1 is easily programmable using known techniques and gRNA design tools known in the art, which means that the assay and assay modules are easily adapted as viral strains emerge and fade in the population.

The embodiments of the signal boost assay described herein provide a reaction mix comprising: first ribonucleoprotein complexes (RNP1s) comprising a first Cas enzyme that exhibits both cis- and trans-cleavage activity and several to many first gRNAs (gRNA1s) where different RNP1s comprise different gRNA1s that are specific for different influenza virus nucleic acids (or other viral or bacterial nucleic acids); a second ribonucleoprotein complex (RNP2) comprising a second Cas enzyme that also exhibits both cis- and trans-cleavage activity and a second gRNA; blocked split activator molecules; and reporter moieties. In the signal boost assay, RNP1 is not activated unless and until a target nucleic acid molecule (here, an influenza virus nucleic acid) is detected.

FIG. 1B is a schematic overview of three exemplary embodiments of methods for performing the signal boost assay on an assay module to detect known and previously unknown influenza viruses. In a first step, first gRNAs (gRNA1s) are designed to several to many influenza virus nucleic acids (101) (RNAs). That is, as shown in FIG. 1A, fifteen gRNA1s are designed to detect different nucleic acids from Influenza A and Influenza B (for fifteen different partitions), with three partitions dedicated to detecting new strains or other nucleic acids of interest, one partition dedicated to detecting SARS-CoV-2, and one partition comprising a control. Designing the gRNA1s may be accomplished using known techniques and gRNA design tools known in the art. At step (102), RNP1s are synthesized with the gRNA1s and a first nucleic acid-guided nuclease of choice. Example II, infra, discloses a method for forming or synthesizing RNPs.

Separately, a sample is obtained 103. A sample can be taken from a nasal swab, a nasopharyngeal swab, oropharyngeal swab, saliva, or other respiratory fluid source. Samples may also be obtained from animal sources (such as swine, cattle, bats, birds) and/or environmental sources such as air, water (including water treatment facilities), surfaces, clinical sites, industrial sites (including food processing sites) and processing facilities. Once the sample is obtained, it is prepared (104) where viruses present in the sample are lysed and the nucleic acids from these viruses are fragmented.

For the two-ribonucleoprotein complex signal boost assay described herein, second ribonucleoprotein complexes (RNP2s) comprising a second guide nucleic acid (i.e., gRNA2) and a second nucleic acid nuclease are synthesized (106). RNP2s are described in detail infra. For most or all of the embodiments of the signal boost assay, the same RNP2 (gRNA2+second nucleic acid-guided nuclease) will be used in all partitions and can be delivered to the assay module in bulk. The RNP2s are formed in the same manner as the RNP1s, and again, Example II, infra, discloses a method for forming or synthesizing RNPs.

In addition to synthesis of the RNP1s and RNP2s, blocked split activator molecules are synthesized (107). As described in detail below, blocked split activator molecules keep the second ribonucleoprotein complexes (RNP2s) which boost the signal of a reporter moiety “locked” unless and until an influenza virus nucleic acid activates the RNP1s. Finally, reporter moieties are synthesized (108). The reporter moieties produce a detectable signal upon induction, as described in detail below.

In method (130), once the sample has been prepared (104) and the signal boost assay components have been synthesized (102, 106, 107 and 108), the different gRNA1s are distributed into partitions at known addresses (131). Typically, one or more RNP1s designed to target influenza virus nucleic acids will be distributed in partitions 1-20—as exemplified in FIG. 1A—with at least one partition comprising a control. The key is that the RNP1s that are in each partition at a particular address are known. Note that as described in this embodiment, the RNP1s are added to each partition in the assay module. As an alternative and in some embodiments, the RNP1s can be formed in the partitions in the assay module. That is, the different gRNA1s can be distributed into partitions of known address, with the first nucleic acid guided nuclease added to each partition under conditions for forming the RNP1s. The number of partitions will depend on the typical volume of a sample and the number of target nucleic acids and controls to be detected. In a typical diagnostic assay where the influenza virus nucleic acids may be at a low concentration (i.e., as low as femtomolar range), the number of partitions will range from 2-50, or from 2-25, or from 2-20. If amplification of the sample is employed, more partitions can be used. Note that although 20 partitions are shown in this FIG. 1A, one of ordinary skill in the art should appreciate given the present disclosure that fewer partitions may be used, detecting fewer influenza A and/or influenza B nucleic acids, or more partitions may be used, detecting, in addition to influenza A and/or influenza B nucleic acids, respiratory viruses that are not pooled, or smaller pools or respiratory viruses and/or bacteria. For example, there may be as few as 6 partitions and as many as 50 partitions, depending on the sample size and the number of nucleic acids being queried.

Once the RNP1s have been distributed into partitions of known address (131), the sample is added to partition region of the assay module (132) in “bulk” or in a “single bolus”—such as from a central “hub” or “sample splitting region” as shown in FIG. 1A—and allowed to contact the RNP1s for, e.g., less than 5 minutes, or less than 2 minutes, or less than 1 minute to facilitate interaction of the nucleic acids in the sample (including the influenza virus nucleic acids, if present) and the different RNP1s in the partitions. Once the sample has had time to interact with the different RNP1s in the partitions, the other signal boost assay components (RNP2s (from 106), blocked split activator molecules (from 107) and reporter moieties (from 108)) are added to each partition in bulk or in a single bolus (133) where reaction conditions are provided for the signal boost assay to take place. As discussed below, even at ambient temperatures of about 16-20° C. or less up to 48° C. the signal boost assay can detect influenza virus nucleic acids present in the sample. In a final step, the signal generated by trans-cleavage of the reporter moieties is detected at each address (134).

Method (135) begins by adding both the RNP1s (from 102) and reporter moieties (from 108) into each partition at known addresses (136) before the sample is added in a single bolus (137) from, e.g., a “hub” region in the assay module. Again, the RNP1s are allowed to interact with the nucleic acids (including the influenza virus nucleic acids) in the sample, followed by the addition of the RNP2s (from 106) and blocked split activator molecules (from 107) (138) in a single bolus, and detection of signal from the reporter moieties (134). Note again, method (135) accomplishes what method 130 does but alters the sequence of adding certain of the assay components and the sample. Note also that both methods (130, 135) deliver both the sample and certain of the assay components “in bulk” or in a single bolus. Method (140) is similar to methods (130) and (135); however, in method (140), all assay components aside from the RNP2s (i.e., RNP1s (from 102), blocked split activator (from 107), and reporter moieties (from 108)) are added to the partitions (with the RNP1s at known addresses) (141) before the sample is added to the partitions (142) in the assay module in a single bolus. In method (140), RNP2 is added to the assay module last in a single bolus (143) followed by detection of signal from the reporter moieties (134). FIG. 1B thus illustrates three exemplary embodiments of varying the sequence of adding the sample and assay components to partitions comprising the RNP1s.

In the methods (130, 135, and 140) just described, an alternative approach is that the gRNA1s may be in the partitions and the first nucleic acid-guided nuclease may be part of the reaction mix of assay components along with the other assay components. In this case, the RNP1s form in the partitions in the presence of the other assay components. Although this alternative is possible, it is preferable that the RNP1s are pre-formed to speed reaction kinetics.

One of ordinary skill in the art given the present disclosure will appreciate that the assay components may be combined in various sequences and configurations, where, e.g., the assay components may be delivered to or reside within the assay module in freeze-dried or lyophilized form as a “bead” or residing on microcarriers. For example, there may be a combined RNP1/RNP2 lyophilized bead rather than two separate lyophilized beads, one for each of RNP1 and RNP2. In addition, one or more of the reaction salts and buffers may reside in a lyophilized bead, along with the blocked split activator molecules and reporter moieties. Alternatively, there may be a “universal bead” comprising all of the assay components. Again, the assay components may be added and distributed in many different configurations as long as different RNP1s are in different partitions.

FIG. 1C shows a simplified exemplary embodiment of a sample splitting assay module and a non-sample splitting assay module. At left in FIG. 1C is an exemplary design of a sample splitting assay module comprising a central well or hub (“sample splitting zone”) where a sample can be distributed, with twenty channels radiating out from the central well, ending in terminal reaction wells. This particular sample splitting assay module is exemplified in the influenza detection module described in relation to FIG. 1A. In this sample splitting assay module, a sample is distributed in the center of the assay module, then aliquots of the sample are driven by positive or negative pressure into the twenty channels and toward, e.g., the terminal reaction wells where the assay components are distributed. Each of the twenty terminal reaction wells comprises a known (and different) RNP1. The other assay components (i.e., RNP2, blocked split activator molecules, and reporter moieties) as described above may reside in the terminal wells with the RNP1s or may be distributed in bulk, with, before or after the sample via a central hub.

At right in FIG. 1C is an exemplary design of a non-sample splitting module comprising twenty-five reaction wells. In this assay module, there is a 5×5 array of reaction wells each comprising a known (and different) RNP1 and the other assay components (i.e., RNP2, blocked split activator molecules, and reporter moieties). A sample is driven in a single bolus or in “bulk” by air displacement using positive or negative pressure across the reaction wells, where the sample is allowed to contact the RNP1s in the partitions to facilitate interaction of the nucleic acids (including the influenza virus nucleic acids, if present) in the sample and the RNP1s in the partitions. Again, the other assay components—RNP2s, blocked split activator molecules and reporter moieties—may reside within the reaction wells with the RNP1s, or may be distributed in bulk across the reaction wells with, before or after the sample.

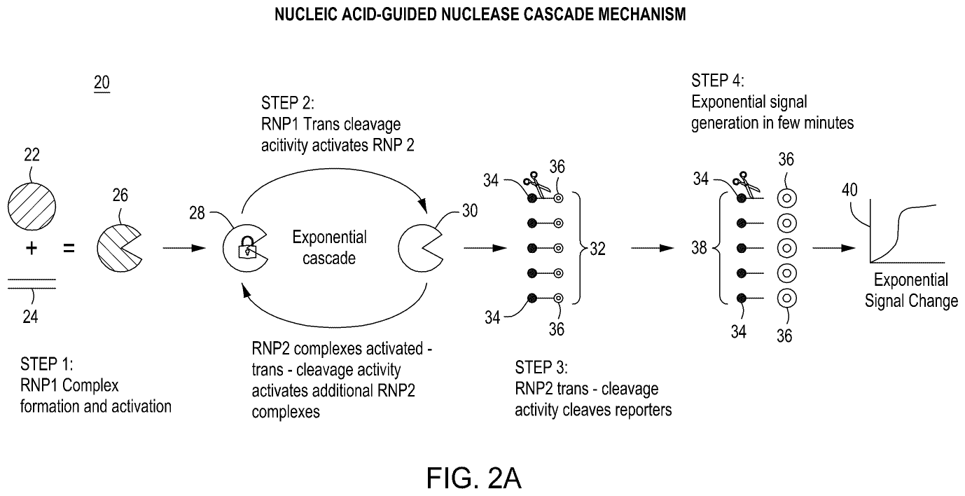

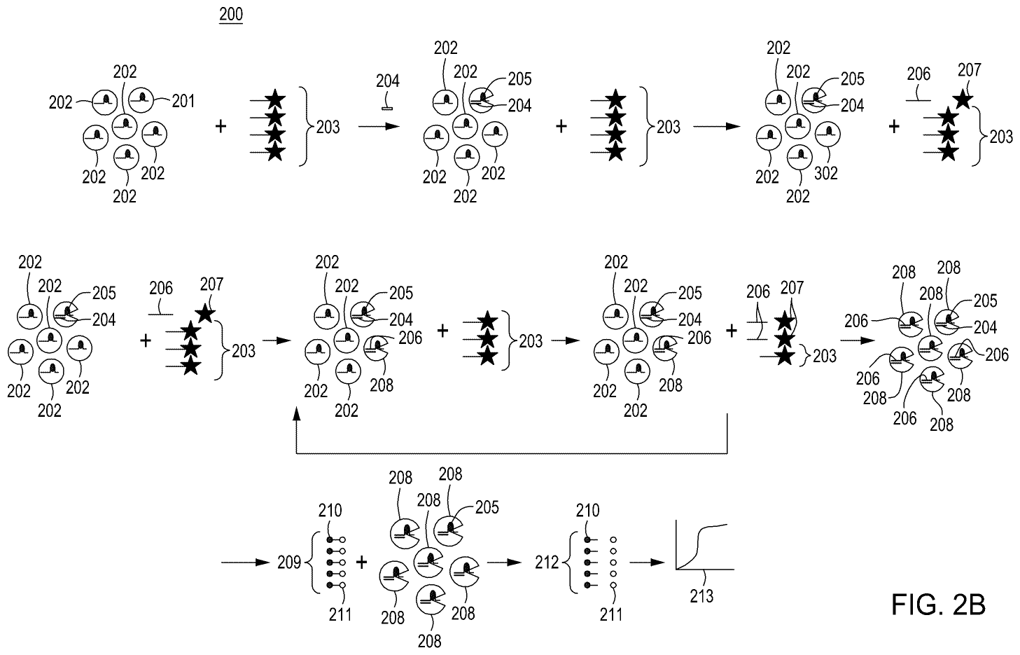

FIG. 2A provides a simplified diagram demonstrating a method (20) of a signal boost assay. The signal boost assay is initiated when a influenza virus nucleic acid (24) binds to and activates a first pre-assembled ribonucleoprotein complex (RNP1) (22). The ribonucleoprotein (RNP) complex comprises a guide RNA (gRNA) and a nucleic acid-guided nuclease, where the gRNA is complexed with the nucleic acid-guided nuclease. The gRNA in RNP1 (i.e., gRNA1), which includes a sequence complementary to one of the influenza virus nucleic acids, guides RNP1 (22) to the influenza virus nucleic acid and hybridizes to it. Typically, preassembled RNP complexes are employed in the reaction mix—as opposed to separate nucleic acid-guided nucleases and gRNAs—to facilitate rapid detection of the influenza virus nucleic acids.

“Activation” of RNP1 refers to activating or initiating trans-cleavage activity of the nucleic acid-guided nuclease in RNP1 (26) by binding of the influenza virus nucleic acid to the gRNA of RNP1. Binding of the influenza virus nucleic acid initiates cis-cleavage activity where the influenza virus nucleic acid is cleaved by the nucleic acid-guided nuclease in RNP1. Binding of the influenza virus nucleic acid to RNP1 also initiates trans-cleavage activity (i.e., multi-turnover activity) of the nucleic acid-guided nuclease in RNP1, where trans-cleavage is indiscriminate, leading to non-sequence-specific cutting of nucleic acid in the reaction mix. This trans-cleavage activity triggers activation of the second ribonucleoprotein complexes (RNP2s) (28) as described in detail below. Each newly activated RNP2 (30) activates more trans-cleavage activity of more RNP2s (28→30), which in turn cleaves more reporter moieties (32). As described in detail below, the reporter moieties (32) may be a synthetic molecule linked or conjugated to a quencher (34) and a fluorophore (36) such as, for example, a probe with a dye label (e.g., FAM or FITC) on the 5′ end and a quencher on the 3′ end. The quencher (34) and fluorophore (36) can be about 20-30 bases apart (or about 10-11 nm apart) or less for effective quenching via fluorescence resonance energy transfer (FRET).

As more RNP2s are unquenched (28→30), more trans-cleavage activity is initiated and more reporter moieties are unquenched; thus, the binding of the influenza virus nucleic acid (24) to RNP1 (22) initiates what becomes a cascade of signal production (40) from the reporter moieties (32→38), which increases exponentially; hence, the term “signal boost.” The signal boost assay thus comprises a single turnover event that triggers a multi-turnover event that then triggers more multi-turnover events in a “cascade.” As described below, the reporter moieties (32) may be provided as molecules that are separate from the other components of the nucleic acid-guided nuclease signal boost assay, or the reporter moieties may be covalently or non-covalently linked to the blocked split activator molecules 6 (i.e., the target molecules for the RNP2).

### Influenza Virus Nucleic Acids

The target nucleic acids of interest in the present context are influenza virus nucleic acids (RNAs), other respiratory virus nucleic acids, bacterial nucleic acids and various controls. The influenza virus nucleic acids may be isolated from a sample by standard laboratory techniques. Non-limiting examples of biological samples include saliva, mucus, a nasal swab, or a nasopharyngeal swab. The source of the sample could be any mammal, such as, but not limited to, a human, primate, monkey, cat, dog, mouse, pig, cow, horse, sheep, and bat. Samples may also be obtained from any other source, such as air, water, soil, surfaces, clinical sites, industrial sites (including food processing sites), agricultural equipment and sites, and commercial samples.

In some embodiments, the target nucleic acids of interest are from one to many infectious agents (e.g., a bacteria, virus, or fungus) that affect mammals, including humans. In the present context, at least some of the target nucleic acids are influenza virus nucleic acids and may be one or more nucleic acid molecules from influenza A and influenza A subtypes, influenza B, PIV 1, PIV 2, PIV 3, or PIV 4. In addition to the influenza virus nucleic acids, testing can also detect RSV, AdV, seasonal CoVs (HKU1, NL63, 229E, OC43), SARS-CoV-2, HMPV, or R/EV.

The signal boost assays described herein are particularly well-suited for simultaneous testing of multiple targets via multiplexed gRNAs as described below. In addition to testing for testing of single matrix, hemagglutinin, neuraminidase, nucleo- and other viral and bacterial protein-related nucleic acids, pools of two to 50 different influenza virus nucleic acids may be detected, e.g., pools of two to 50, or two to 30, or two to 25, or two to 20, or two to 10 influenza virus nucleic acids.

The methods described herein do not require the target nucleic acids of interest to be DNA, and in fact it is specifically contemplated that the influenza virus nucleic acids to be detected are RNA.

### Nucleic Acid-Guided Nucleases

The signal boost assays comprise nucleic acid-guided nucleases in the reaction mix, either provided as a protein, a coding sequence for the protein, or, in most embodiments, in a ribonucleoprotein (RNP) complex. In some embodiments, the one or more nucleic acid-guided nucleases in the reaction mix may be, for example, a Cas nucleic acid-guided nuclease. Any nucleic acid-guided nuclease having trans-cleavage activity may be employed, and the same nucleic acid-guided nuclease may be used for both RNP complexes or different nucleic acid-guided nucleases may be used in RNP1 and RNP2. For example, RNP1 and RNP2 may both comprise Cas12a nucleic acid-guided nucleases, or RNP1 may comprise a Cas13 nucleic acid-guided nuclease and RNP2 may comprise a Cas12a nucleic acid-guided nuclease or vice versa. Note that trans-cleavage activity is not triggered unless and until an influenza virus nucleic acid target binds to RNP1 or an unblocked split activator molecule binds to RNP2. Nucleic acid-guided nucleases include Type V and Type VI nucleic acid-guided nucleases, as well as nucleic acid-guided nucleases that comprise a RuvC nuclease domain or a RuvC-like nuclease domain but lack an HNH nuclease domain. Nucleic acid-guided nucleases with these properties are reviewed in Makarova and Koonin, Methods Mol. Biol., 1311:47-75 (2015) and Koonin, et al., Current Opinion in Microbiology, 37:67-78 (2020) and updated databases of nucleic acid-guided nucleases and nuclease systems that include newly-discovered systems include BioGRID ORCS (orcs: thebiogrid.org); GenomeCRISPR (genomecrispr.org); Plant Genome Editing Database (plantcrispr.org) and CRISPRCasFinder (crispercas.i2bc.paris-saclay.fr).

The type of nucleic acid-guided nuclease utilized in the method of detection depends on the type of nucleic acid to be detected. For example, a DNA nucleic acid-guided nuclease (e.g., a Cas12a, Cas14a, or Cas3) should be utilized if the target nucleic acid is a DNA molecule, and an RNA nucleic acid-guided nuclease (e.g., Cas13a or Cas12g) should be utilized if the target nucleic acid (such as an influenza virus nucleic acid) is an RNA molecule. Exemplary nucleic acid-guided nucleases include, but are not limited to, Cas RNA-guided DNA nucleic acid-guided nucleases, such as Cas3, Cas12a (e.g., AsCas12a, LbCas12a), Cas12b, Cas12c, Cas12d, Cas12e, Cas14, Cas12h, Cas12i, and Cas12j; Cas RNA-guided RNA nucleic acid-guided nucleases, such as Cas13a (LbaCas13, LbuCas13, LwaCas13), Cas13b (e.g., CccaCas13b, PsmCas13b), and Cas12g; and any other nucleic acid-(DNA, RNA, or cDNA) targeting nucleic acid-guided nuclease with collateral trans-cleavage activity. In embodiments where only influenza virus nucleic acids are detected, the nucleic acid-guided nucleases employed are RNA nucleic acid nucleases (i.e., nucleases that cleave RNA nucleic acids). In embodiments where influenza virus nucleic acids and other nucleic acids are detected, if the other nucleic acids are DNA target nucleic acids, then some of the RNP1s will comprise a DNA nucleic acid-guided nuclease.

### Guide RNA (gRNA)

The present disclosure detects influenza virus nucleic acids (and other viral and bacterial agents associated with respiratory infections) via a reaction mixture containing at least two guide RNAs (gRNAs) (i.e., gRNA1 and gRNA2) each incorporated into a different RNP complex (i.e., RNP1 and RNP2). Suitable gRNAs include at least one crRNA region to enable specificity in every reaction. The gRNA1s of the RNP1s are specific to the influenza virus nucleic acids (and other agents) and the gRNA2s of the RNP2s are specific to an unblocked split activator molecules, described in detail below. As will be clear given the description below, an advantageous feature of the signal boost assay is that, with the exception of the gRNA1s in the RNP1s (i.e., the gRNAs specific to the influenza virus nucleic acids), the signal boost assay components can stay the same (i.e., are identical or substantially identical) no matter what influenza virus nucleic acids, controls, etc., are being detected, and the gRNA1s in the RNP1s are easily reprogrammable using known techniques and gRNA design tools.

Like the nucleic acid-guided nuclease, the gRNA is preferably provided in the signal boost assay reaction mix in a preassembled RNP, as an RNA molecule, or may also be provided as a DNA sequence to be transcribed, in, e.g., a vector backbone; however, providing the gRNA in a pre-assembled RNP complex (i.e., RNP1 or RNP2) is preferred if rapid kinetics are preferred. Guide RNAs are generally about 20 nucleotides to about 300 nucleotides in length and may contain a spacer sequence containing a plurality of bases and complementary to a protospacer sequence in the target sequence. The gRNA spacer sequence may be 50%, 60%, 75%, 80%, 85%, 90%, 95%, 97.5%, 98%, 99%, or more complementary to its intended influenza virus nucleic acid.

The gRNA of RNP1 is capable of binding to the nucleic acid-guided nuclease of RNP1 to perform cis-cleavage of an influenza (or other respiratory) virus nucleic acid, where the binding also triggers non-sequence specific trans-cleavage of other molecules in the reaction mix. Guide RNAs include any polynucleotide sequence having sufficient complementarity with an influenza virus nucleic acid (or RNP2 target sequences which are unblocked blocked split activator molecules).

In any of the foregoing embodiments, the gRNA may be a modified or non-naturally occurring nucleic acid molecule. In some embodiments, the gRNAs of the disclosure may further comprise a locked nucleic acid (LNA), a bridged nucleic acid (BNA), and/or a peptide nucleic acid (PNA). By way of further example, a modified nucleic acid molecule may comprise a modified or non-naturally occurring nucleoside, nucleotide, and/or internucleoside linkage, such as a 2′-O-methyl (2′-O-Me) modified nucleoside, a 2′-fluoro (2′-F) modified nucleoside, and a phosphorothioate (PS) bond, or any other nucleic acid molecule modifications described herein.

### Ribonucleoprotein (RNP) Complexes

Although the signal boost assay “reaction mix” or “reaction mixture” may comprise separate nucleic acid-guided nucleases and gRNAs (or coding sequences therefor), the signal boost assays preferably comprise preassembled ribonucleoprotein complexes (RNPs) in the reaction mix, allowing for faster detection kinetics. The present signal boost assay employs at least two types of RNP complexes—RNP1 and RNP2—each type containing a nucleic acid-guided nuclease and a gRNA. RNP1 and RNP2 may comprise the same nucleic acid-guided nuclease or may comprise different nucleic acid-guided nucleases; however, the gRNAs in RNP1 and RNP2 are different and are configured to detect different nucleic acids. Further, as described above, there will be at least two and in some embodiments tens of different RNP1s. In the example shown in FIG. 1A, there are fifteen different influenza virus nucleic acids to be tested, and thus at least fifteen different influenza virus RNP1s in addition to the RNPI(s) specific for SARS-CoV-2, other respiratory viruses and bacterial pathogens are present. In some embodiments, the reaction mixture contains about 1 fM to about 10 μM of a given RNP1, or about 1 pM to about 1 μM of a given RNP1, or about 10 pM to about 500 pM of a given RNP1. In some embodiments the reaction mixture contains about 6×10⁴ to about 6×10¹² complexes per microliter (μl) of a given RNP1, or about 6×10⁶ to about 6×10¹⁰ complexes per microliter (μl) of a given RNP1. In some embodiments, the reaction mixture contains about 1 fM to about 500 μM of a given RNP2, or about 1 pM to about 250 μM of a given RNP2, or about 10 pM to about 100 μM of a given RNP2. In some embodiments the reaction mixture contains about 6×10⁴ to about 6×10¹² complexes per microliter (μl) of a given RNP2 or about 6×10⁶ to about 6×10¹² complexes per microliter (μl) of a given RNP2. See Example II below describing preassembling RNPs.

In any of the embodiments of the disclosure, 1 to about 50 different RNP1s may be used to interrogate target nucleic acids (e.g., 1, 2, 3, 4, 5, 6, 7, 8, 9, 10, 11, 12, 13, 14, 15, 16, 27, 28, 19, 20, 21, 22, 23, 24, 25, 50 or more RNP1s), where different RNP1s comprise different gRNA polynucleotide sequences, with some partitions comprising a single species of RNP1 and some partitions comprising two to several to many pooled RNP1 species. Additionally, as described above in relation to FIG. 1A, more than one RNP1 may be present for the purpose of targeting one influenza virus nucleic acid from each of many sources or more than one (e.g., several to many) RNP1s may be present for the purpose of targeting more than one influenza virus nucleic acid from a single source virus.

The disclosed methods may include a partition containing a single gRNA1, or more than two different gRNA1s, more than three different gRNA1s, more than four different gRNA1s, more than five different gRNA1s, more than six different gRNA1s, more than seven different gRNA1s, more than eight different gRNA1s, more than nine different gRNA1s, more than ten different gRNAs, more than eleven different gRNA1s, more than twelve different gRNA1s, more than thirteen different gRNA1s, more than fourteen different gRNA1s, more than fifteen different gRNA1s, more than sixteen different gRNA1s, more than seventeen different gRNA1s, more than eighteen different gRNA1s, more than nineteen different gRNA1s, more than twenty different gRNA1s, more than twenty-one different gRNA1s, more than twenty-two different gRNA1s, more than twenty-three different gRNA1s, more than twenty-four different gRNA1s, more than twenty-five different gRNA1s, more than fifty different gRNA1s, or more than one hundred different gRNA1s or more.

### Blocked Split Activator Molecules

Blocked split activator molecules typically have a low binding affinity, or high dissociation constant (K_{d}) in relation to binding to RNP2 and may be referred to herein as a high K_{d} nucleic acid molecule. In the context of the present disclosure, the binding of blocked or unblocked split activator molecules to RNP2 have low K_{d} values ranging from about 100 fM to about 1 aM or lower (e.g., 100 zM). High K_{d} values range from 100 nM to about 10-100 10 mM; thus, high K_{d} values are about 10⁵−, 10⁶−, 10⁷−, 10⁸−, 10⁹− to 10¹⁰− fold or higher as compared to low K_{d} values.

The blocked split activator molecules (high K_{d} molecules) described herein can be converted into unblocked split activator molecules (low K_{d} molecules—also in relation to binding to RNP2) via cleavage of nuclease-cleavable regions (e.g., via active RNP1s and RNP2s). The unblocked split activator molecule has a much higher binding affinity for the gRNA in the RNP2 than does the blocked split activator molecule.

Once the unblocked split activator molecule is bound to RNP2, the RNP2 activation triggers trans-cleavage activity, which in turn leads to more RNP2 activation by further cleaving blocked split activator molecules to produce more unblocked split activator molecules, resulting in a positive feedback loop.

### Reporter Moieties

The signal boost assay detects an influenza virus nucleic acid via detection of a signal generated in the reaction mix typically by a reporter moiety. In many embodiments the detection of the influenza virus nucleic acids occurs within thirty minutes of assay initiation. Reporter moieties can comprise DNA, RNA, a chimera of DNA and RNA, and can be single stranded, double stranded, or a moiety that is a combination of single stranded portions and double stranded portions. In the context of detecting influenza virus nucleic acids, at least some of the reporter moieties typically comprise RNA.

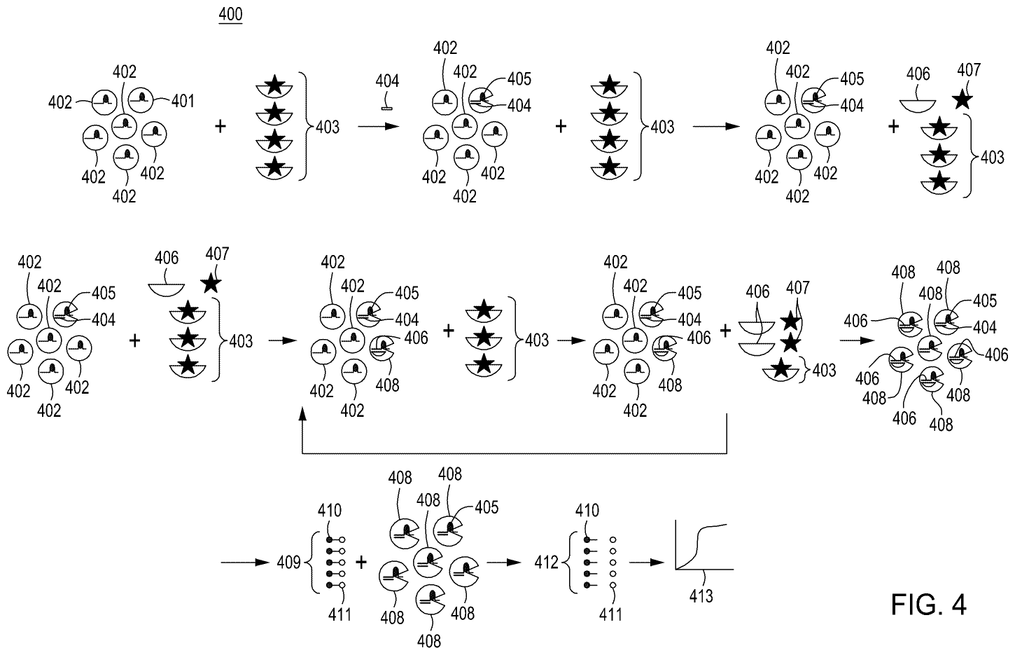

Depending on the type of reporter moiety used, trans-and/or cis-cleavage by the nucleic acid-guided nuclease in RNP2 releases a signal. In some embodiments, trans-cleavage of stand-alone reporter moieties (e.g., not bound to any blocked split activator molecules) may generate signal changes at rates that are proportional to the cleavage rate, as new RNP2s are activated over time (shown at bottom in FIGS. 2B and 4). Trans-cleavage by either an activated RNP1 or an activated RNP2 may release a signal although the vast majority of trans-cleavage of the reporter moieties is due to the trans-cleavage activity of RNP2. In alternative embodiments and preferably, the reporter moiety may be bound to the blocked split activator molecule, where trans-cleavage of the blocked split activator molecule and conversion to an unblocked split activator molecule generates signal changes at rates that are proportional to the cleavage rate as new RNP2s are activated over time.

The reporter moiety may be a synthetic molecule linked or conjugated to a reporter and quencher such as, for example, a TaqMan probe with a dye label (e.g., FAM or FITC) on the 5′ end and a quencher on the 3′ end. The reporter and quencher may be about 20-30 bases apart or less (i.e., 10-11 nm apart or less) for effective quenching via fluorescence resonance energy transfer (FRET). Alternatively, signal generation may occur through different mechanisms. Other detectable moieties, labels, or reporters can also be used to detect an influenza virus nucleic acid as described herein. Reporter moieties can be labeled in a variety of ways, including direct or indirect attachment of a detectable moiety such as a fluorescent moiety, hapten, or colorimetric moiety.

Examples of detectable moieties include various radioactive moieties, enzymes, prosthetic groups, fluorescent markers, luminescent markers, bioluminescent markers, metal particles, and protein-protein binding pairs, e.g., protein-antibody binding pairs. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, and phycoerythrin. Examples of bioluminescent markers include, but are not limited to, luciferase (e.g., bacterial, firefly, click beetle and the like), luciferin, and acquorin. Examples of enzyme systems having visually detectable signals include, but are not limited to, galactosidases, glucorinidases, phosphatases, peroxidases, and cholinesterases. Identifiable markers also include radioactive elements such as ¹²⁵1, ³⁵S, ¹⁴C, or ³H. Reporters can also include a change in pH or charge of the signal boost assay reaction mix.

The methods used to detect the generated signal will depend on the reporter moiety or moieties used. For example, a radioactive label can be detected using a scintillation counter, photographic film as in autoradiography, or storage phosphor imaging. Fluorescent labels can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Enzymatic labels can be detected by providing the appropriate substrates for the enzyme and detecting the resulting reaction product. Simple colorimetric labels can be detected by observing the color associated with the label. When pairs of fluorophores are used in an assay, fluorophores are chosen that have distinct emission patterns (wavelengths) so that they can be easily distinguished.

Single-stranded, double-stranded or reporter moieties comprising both single- and double-stranded portions can be introduced to show a signal change proportional to the cleavage rate, which increases with every new activated RNP2 complex over time. For example, the method of detecting an influenza virus nucleic acid in a sample using a signal boost assay as described herein can involve contacting the reaction mix with a labeled detection ssRNA containing a fluorescent resonance energy transfer (FRET) pair, a quencher/phosphor pair, or both. A FRET pair consists of a donor chromophore and an acceptor chromophore, where the acceptor chromophore may be a quencher molecule. FRET pairs (donor/acceptor) suitable for use include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5, Texas Red/DABCYL, BODIPY/DABCYL, Lucifer yellow/DABCYL, coumarin/DABCYL, and fluorescein/LC Red 705. In addition, a fluorophore/quantum dot donor/acceptor pair can be used. EDANS is (5-((2-Aminoethyl)amino) aphthalenc-1-sulfonic acid); IAEDANS is 5-({2-[(iodoacetyl)amino]ethyl}amino) naphthalene-1-sulfonic acid); DABCYL is 4-(4-dimethylaminophenyl)diazenylbenzoic acid. Useful quenchers include, but are not limited to, BHQ, DABCYL, QSY 7 and QSY 33.

In any of the foregoing embodiments, the reporter moiety may comprise one or more modified nucleic acid molecules, containing a modified nucleoside or nucleotide. In some embodiments the modified nucleoside or nucleotide is chosen from 2′-O-methyl (2′-O-Me) modified nucleoside, a 2′-fluoro (2′-F) modified nucleoside, and a phosphorothioate (PS) bond, or any other nucleic acid molecule modifications described below.

### Nucleic Acid Modifications

For any of the nucleic acid molecules described herein (e.g., blocked split activator molecules, gRNAs, and reporter moieties), the nucleic acid molecules may be used in a wholly or partially modified form. Typically, modifications to the blocked split activator molecules, gRNAs, and reporter moieties described herein are introduced to optimize the molecule's biophysical properties (e.g., increasing nucleic acid-guided nuclease resistance and/or increasing thermal stability). Modifications typically are achieved by the incorporation of, for example, one or more alternative nucleosides, alternative sugar moieties, and/or alternative internucleoside linkages.

For example, one or more of the signal boost assay components may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenineand guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The nucleic acid molecules described herein (e.g., blocked split activator molecules, gRNAs, and reporter molecules) may also include nucleobases in which the purine or pyrimidine base is replaced with other heterocycles, for example 7-deaza-adenine, 7-deazaguanosine, 2-aminopyridine, and/or 2-pyridone. Further modification of the nucleic acid molecules described herein may include nucleobases disclosed in U.S. Pat. No. 3,687,808; Kroschwitz, ed., The Concise Encyclopedia of Polymer Science and Engineering, NY, John Wiley & Sons, 1990, pp. 858-859; Englisch, et al., Angewandte Chemie, 30:613 (1991); and Sanghvi, Chapter 16, Antisense Research and Applications, CRC Press, Gait, ed., 1993, pp. 289-302.

In addition to or as an alternative to nucleoside modifications, the signal boost assay components may comprise 2′ sugar modifications, including 2′-O-methyl (2′-O—Me), 2′-methoxyethoxy (2′-O—CH₂CH₂OCH₃, also known as 2′-O—(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can modify the nucleic acid molecules described herein (i.e., blocked split activator molecules, gRNAs, and reporter molecules) may include all possible orientations of OH; F; O-, S-, or N-alkyl (mono- or di-); O-, S-, or N-alkenyl (mono-or di-); O-, S- or N-alkynyl (mono- or di-); or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O-CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

Finally, modifications to the signal boost assay components may comprise internucleoside modifications such phosphorothioates, phosphorodithioates, phosphotriesters, aminoalkylphosphotriesters, methyl and other alkyl phosphonates including 3′-alkylenc phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### The Signal Boost Assay Employing Blocked Nucleic Acid Molecules

As described above in relation to FIG. 2A, the signal boost assay is performed in partitions in assay modules, where each partition comprises one or more specific, known RNP1s. FIG. 2A, described above, depicts the signal boost assay generally. A specific embodiment of the signal boost assay utilizing blocked nucleic acid molecules is depicted in FIG. 2B and described in detail below. In this embodiment, a blocked nucleic acid is used to prevent the activation of RNP2 in the absence of an influenza virus nucleic acid. The method in FIG. 2B begins with providing the signal boost assay components RNP1 (201), RNP2 (202) and blocked nucleic acid molecules (203). RNP1 (201) comprises a gRNA specific for an influenza virus nucleic acid and a nucleic acid-guided nuclease (Cas 13a or Cas12g for an RNA influenza virus nucleic acid) and RNP2 (202) comprises a gRNA specific for an unblocked nucleic acid molecule and a nucleic acid-guided nuclease (again, e.g., Cas12a or Cas 14 for a DNA unblocked nucleic acid molecule or a Cas 13a or Cas12g for an RNA unblocked nucleic acid molecule). As described above, the nucleic acid-guided nucleases in RNP1 (201) and RNP2 (202) can be the same or different depending on the type of influenza virus nucleic acid and unblocked nucleic acid molecule. What is key, however, is that the nucleic acid-guided nucleases in RNP1 and RNP2 are activated to have trans-cleavage activity following an influenza virus nucleic acid or unblocked nucleic acid molecule binding.

In a first step, a sample comprising an influenza virus nucleic acid (204) is added to the signal boost assay reaction mix. Keep in mind that although shown as a single reaction, the method depicted in FIG. 2B is performed in several to many partitions in parallel (e.g., in the context of the influenza panel discussed in relation to FIG. 1A, 20 partitions), where each partition comprises one or more different RNP1s. Again, there may be and typically will be pooled RNP1s within a single partition for, e.g., testing for two or more variants in at least the hemagglutinin subtype; for testing for two or more variants in the seasonal coronaviruses (CoVs) (OC43, NL63, HKU-1, 229E), SARS CoV-2, AdV, RSV, PIV 1,2,3,4, HMPV, and R/EV and for testing for two or more variants in each bacterial pathogen. That is, a single partition may comprise many different RNP1s that detect different variants or subvariants of, e.g., SARS-CoV-2. Additionally, there may be pooled RNP1s in a single partition to detect two or more of, e.g., coronaviruses (CoVs) (OC43, NL63, HKU-1, 229E), SARS CoV-2, AdV, RSV, PIV 1,2,3,4, HMPV, and R/EV; or there may be pooled RNP1s in a single partition to detect two or more bacterial pathogens.

The influenza virus nucleic acid (204) combines with and activates RNP1 (205) but does not interact with or activate RNP2 (202). Once activated, RNP1 binds the influenza virus nucleic acid (204) and cuts the influenza virus nucleic acid (204) via sequence-specific cis-cleavage while activating non-specific trans-cleavage of other nucleic acids present in the reaction mix, including the blocked nucleic acid molecules (203). At least one of the blocked nucleic acid molecules (203) becomes an unblocked nucleic acid molecule (206) when the blocking moiety (207) is removed. As described above, “blocking moiety” may refer to nucleoside modifications, topographical configurations such as secondary structures, and/or structural modifications.

Once at least one of the blocked nucleic acid molecules (203) is unblocked, the unblocked nucleic acid molecule (206) can then bind to and activate an RNP2 (208). Because the nucleic acid-guided nucleases in the RNP1s (205) and RNP2s (208) have both cis- and trans-cleavage activity, the trans-cleavage activity causes more blocked nucleic acid molecules (203) become unblocked nucleic acid molecules (203→206) triggering activation of even more RNP2s (208) and more trans-cleavage activity in a cascade. FIG. 2B at bottom depicts the concurrent activation of reporter moieties. Intact reporter moieties (209) comprise a quencher (210) and a fluorophore (211) linked by a nucleic acid sequence. As described above in relation to FIG. 2A, the reporter moieties are also subject to trans-cleavage by activated RNP1 (205) and RNP2 (208). The intact reporter moieties (209) become activated reporter moieties (212) when the quencher (210) is separated from the fluorophore (211), emitting a fluorescent signal (213). Signal strength increases rapidly as more blocked nucleic acid molecules (203) become unblocked nucleic acid molecules (206) triggering cis-cleavage activity of more RNP2s (208) and thus more trans-cleavage activity of the reporter moieties (209). Again, the reporter moieties are shown here as separate molecules from the blocked nucleic acid molecules, but other configurations may be employed. One particularly advantageous feature of the signal boost assay is that, with the exception of the gRNA in the RNP1 (gRNA1), the signal boost assay components are modular in the sense that the components can stay the same no matter what target nucleic acids of interest are being detected.

A blocked nucleic acid molecule may be single-stranded or double-stranded, circular or linear, and may further contain a partially hybridized nucleic acid sequence containing cleavable secondary loop structures, as exemplified in FIG. 2B. Such blocked nucleic acid molecules typically have a low binding affinity, or high dissociation constant (K_{d}) in relation to binding to RNP2 and may be referred to herein as a high K_{d} nucleic acid molecule. In the context of the present disclosure, the binding of blocked or unblocked nucleic acid molecules to RNP2, low K_{d} values range from about 100 fM to about 1 aM or lower (e.g., 100 zM) and high K_{d} values are in the range of 100 nM to about 10-100 10 mM and thus are about 10⁵-, 10⁶-, 10⁷-, 10⁸-, 10⁹- to 10¹⁰-fold or higher as compared to low K_{d} values. Of course, the ideal blocked nucleic acid molecule would have an “infinite K_{d}.”

The blocked nucleic acid molecules (high K_{d} molecules) described herein can be converted into unblocked nucleic acid molecules (low K_{d} molecules—also in relation to binding to RNP2) via cleavage of nuclease-cleavable regions (e.g., via active RNP1s and RNP2s). The unblocked nucleic acid molecule has a higher binding affinity for the gRNA in RNP2 than does the blocked nucleic acid molecule.

Once the unblocked nucleic acid molecule is bound to RNP2, the RNP2 activation triggers trans-cleavage activity, which in turn leads to more RNP2 activation by further cleaving blocked nucleic acid molecules, resulting in a positive feedback loop or cascade.

In embodiments where blocked nucleic acid molecules are linear and/or form a secondary structure, the blocked nucleic acid molecules may be single-stranded (ss) or double-stranded (ds) and contain a first nucleotide sequence and a second nucleotide sequence. The first nucleotide sequence has sufficient complementarity to hybridize to a gRNA of RNP2, and the second nucleotide sequence does not. The first and second nucleotide sequences of a blocked nucleic acid molecule may be on the same nucleic acid molecule (e.g., for single-strand embodiments) or on separate nucleic acid molecules (e.g., for double-strand embodiments). Trans-cleavage (e.g., via RNP1 or RNP2) of the second nucleotide sequence converts the blocked nucleic acid molecule to a single-strand unblocked nucleic acid molecule. The unblocked nucleic acid molecule contains only the first nucleotide sequence, which has sufficient complementarity to hybridize to the gRNA of RNP2, thereby activating the trans-cleavage activity of RNP2.

The second nucleotide sequence at least partially hybridizes to the first nucleotide sequence, resulting in a secondary structure containing at least one loop (e.g., hairpin loops, tetraloops, pseudoknots, junctions, kissing hairpins, internal loops, bulges, and multibranch loops). Such loops block the nucleic acid molecule from binding or incorporating into an RNP complex thereby initiating cis-or trans-cleavage. Again, in some embodiments, the blocked nucleic acid molecules are configured as described in see U.S. Pat. Nos. 11,693,520; 11,702,686; 11,821,025; 11,970,730; 11,987,839; 11,884,921; 11,820,983; 11,859,182; 11,884,922; and 11,946,052.

Nucleotide mismatches can be introduced in double-strand regions of the blocked nucleic acid molecules to reduce the melting temperature (Tₘ) of the region (i.e., “clamp”) such that once the loop is cleaved, the double-strand segment is unstable and dehybridizes rapidly. The percentage of nucleotide mismatches of a given region may vary between 0% and 50%; however, the maximum number of nucleotide mismatches is limited to a number where the secondary loop structure still forms. In other words, the number of hybridized bases can be less than or equal to the length of each double-strand segment and vary based on number of mismatches introduced.

In any of the foregoing embodiments, the blocked nucleic acid molecules of the disclosure may further contain a reporter moiety attached thereto such that cleavage of the blocked nucleic acid releases a signal from the reporter moiety.

Also, in any of the foregoing embodiments, the blocked nucleic acid molecule may be a modified or non-naturally occurring nucleic acid molecule. In some embodiments, the blocked nucleic acid molecules of the disclosure may further contain a locked nucleic acid (LNA), a bridged nucleic acid (BNA), and/or a peptide nucleic acid (PNA). The blocked nucleic acid molecule may contain a modified or non-naturally occurring nucleoside, nucleotide, and/or internucleoside linkage, such as a 2′-O-methyl(2′-O-Me) modified nucleoside, a 2′-fluoro(2′-F) modified nucleoside, and a phosphorothioate (PS) bond, any other nucleic acid molecule modifications described above, and any combination thereof.

### The Signal Boosting Cascade Assay Employing Blocked Split Activator Molecules

In an improved alternative to the signal boost cascade assay described above utilizing blocked nucleic acid molecules, there is a signal boosting cascade assay employing blocked split activator molecules. The signal boost cascade assay utilizing blocked split activator molecules has been designed to reduce false positive signals resulting from undesired, non-specific unblocking of blocked nucleic acid molecules by configuring them as blocked split activator molecules. Non-specific unblocking of the blocked nucleic acid molecules leads to non-specific activation of RNP2 in the absence of a target nucleic acid of interest.

In the “correct” pathway for unblocking a blocked nucleic acid molecule, unblocking is due to trans-cleavage of single-strand regions of a blocked nucleic acid molecule leading to activation of RNP2. However, in a “failure” pathway, unblocking of the blocked nucleic acid molecule is due not to trans-cleavage of the blocked nucleic acid molecule and dissociation of the non-target strand oligonucleotide segments from the target strand, but instead is due to erroneous enzyme-mediated unwinding of the blocked nucleic acid molecule by RNP2.

The unwinding of the blocked nucleic acid molecule may be triggered by the presence of a PAM in the blocked nucleic acid molecule; however, even in blocked nucleic acid molecules lacking a PAM sequence unwinding can take place simply by the sequence complementarity between the blocked nucleic acid molecule (i.e., target molecule for RNP2) and gRNA2 which leads to DNA unpairing of the blocked nucleic acid molecule, R-loop formation, and subsequent activation of RNP2. This erroneous activation of RNP2 triggers trans-cleavage activity of RNP2 and subsequent signal generation. In this failure pathway, a target nucleic acid of interest is not present and RNP1 trans-cleavage activity has not been activated, yet RNP2 becomes activated leading to a false positive signal. A solution, as described herein, involves configuring the blocked nucleic acid molecule as a blocked split activator molecule to maintain a tight “lock” on RNP2 in the absence of trans-cleavage activity triggered by the binding of a target nucleic acid of interest to RNP1—thereby circumventing the failure pathway—but then to “reconstitute” the target portion of the unblocked split activator molecule once 3′, 5′, or 3′ and 5′ extensions of the target portion of the split activator has been trans-cleaved and removed.

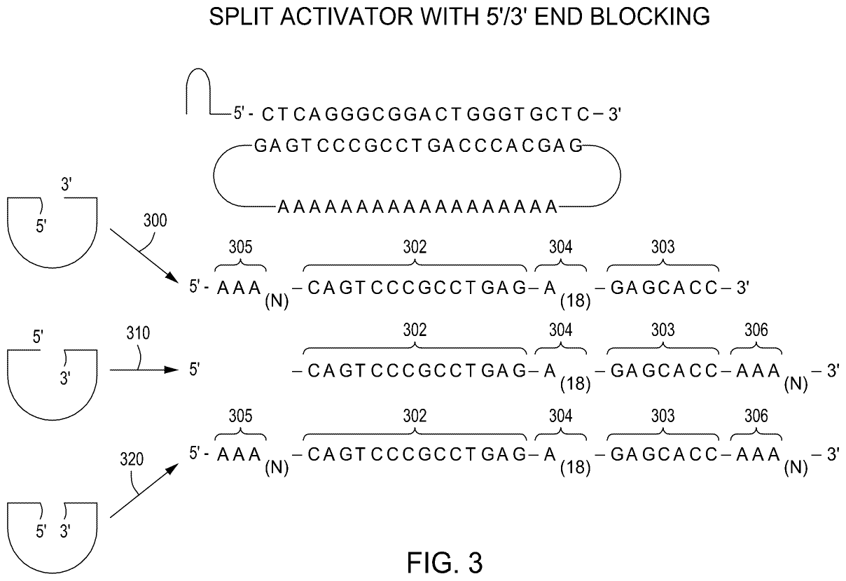

FIG. 3 is a simplified diagram of an exemplary gRNA2 sequence and three exemplary blocked split activator molecules. At top of FIG. 3 is a gRNA with a crRNA sequence: 5′-CTCAGGGCGGACTGGGTGCTC-3′ [SEQ ID NO: 1]; and an unblocked “reconstituted” split activator sequence: 5′-CAGTCCCGCCTGAG-A ₍₁₈₎-GAGCACC -3′ [SEQ ID NO:2]. The A₍₁₈₎ here serves as a linker between the split portions of the target portion of the unblocked split activator molecule. There are three exemplary blocked split activator molecules in the center of FIG. 3, each split with fourteen nucleotides at the 5′ end of the blocked split activator molecule and with seven nucleotides at the 3′ end of the blocked split activator molecule.

The first exemplary blocked split activator molecule (300) is blocked with adenine nucleotides at the 5′ end, comprising the sequence 5′-AAA_{(N)}-CAGTCCCGCCTGAG-A₍₁₈₎-GAGCACC-3′ [SEQ ID NO:3]; the second exemplary blocked split activator molecule (310) is blocked with adenine nucleotides at the 3′ end, comprising the sequence 5′-CAGTCCCGCCTGAG-A₍₁₈₎-GAGCACC-AAA_{(N)}-3′ [SEQ ID NO: 4]; and the third exemplary blocked split activator molecule (320) is blocked with adenine nucleotides at both the 5′ and the 3′ end, comprising the sequence 5′-AAA_{(N)}-CAGTCCCGCCTGAG-A₍₁₈₎-GAGCACC-AAA_{(N)}-3′ [SEQ ID NO:5]. In addition, simplified cartoons of these exemplary blocked split activator molecules are shown at left in FIG. 4. The first blocked split activator molecule (300) comprises four regions: a 5′ target portion (302); a 3′ target portion (303); a linker (304); and a 5′ block (305). The second blocked split activator molecule (310) comprises four regions: a 5′ target portion (302); a 3′ target portion (303); a linker (304); and a 3′ block (306). The third blocked split activator molecule (320) comprises five regions: a 5′ target portion (302); a 3′ target portion (303); a linker (304); a 5′ block (305); and a 3′ block (306).

The blocking moiety on each exemplary blocked split activator molecule shown is a sequence of adenine nucleotides. It has been determined that even one additional nucleotide at the 5′ or 3′ end of the split activator molecules will prevent activation of RNP2. Here, “AAA_{(N)}” is used as an exemplary block; however, even one nucleotide A, T, U, C, or G may be used, and up to eight additional nucleotides of any sequence may be used, with the caveat that ribonucleotides should be used if the nucleic acid-guided nucleases in RNP1 and RNP2 are RNA-cleaving nucleic acid-guided nucleases, deoxyribonucleotides should be used if the nucleic acid-guided nucleases in RNP1 and RNP2 are DNA-cleaving nucleic acid-guided nucleases, and a mix of blocked split activator molecules should be used-some with ribonucleotides and some with deoxyribonucleotides—if one of the nucleic acid-guided nucleases in RNP1 or RNP2 is an RNA-cleaving nucleic acid-guided nuclease and the other nucleic acid-guided nuclease is a DNA-cleaving nucleic acid-guided nuclease. Note that use of an additional nucleotide at the 5′ or 3′ end of the split activator molecule will prevent activation of RNP2; however, in some embodiments, it may be desired to used one or more additional nucleotides at both the 5′ and 3′ end of the blocked split activator molecules depending on preferred kinetics.

In this example, the target portion of the blocked split activator molecules are 21 nucleotides in length and have been split 14/7; however, the target portion may vary from 20 to 30 nucleotides in length and be split, e.g., 14/6, 13/7, 12/8, 11/9, 10/10, 9/11, 8/12, 7/13, or 6/14; or 14/7, 13/8, 12/9, 11/10, 10/11, 9/12, 8/13, 7/14, or 6/15; or 14/8, 13/9, 12/10, 11/11, 10/12, 9/13, 8/14, 7/15, or 6/16; or 14/9, 13/10, 12/11, 11/12, 10/13, 9/14, 8/15, 7/16, or 6/17; or 14/10, 13/11, 12/12, 11/13, 10/14, 9/15, 8/16, 7/17, or 6/18; or 14/11, 13/12, 12/13, 11/14, 10/15, 9/16, 8/17, 7/18, or 6/19; or 14/12, 13/13, 12/14, 11/15, 10/16, 9/17, 8/18, 7/19, or 6/20; or 14/13, 13/14, 12/15, 11/16, 10/17, 9/18, 8/19, 7/20, or 6/21; or 14/14, 13/15, 12/16, 11/17, 10/18, 9/19, 8/20, 7/21, or 6/22; or 14/15, 13/16, 12/17, 11/18, 10/19, 9/20, 8/21, 7/22, or 6/23; or 14/16, 13/17, 12/18, 11/19, 10/20, 9/21, 8/22, 7/23, or 6/24. It has been found that having a 5′ portion of the split exceeding 14 nucleotides compromises the block (data not shown), suggesting that the first 15 or so 5′ nucleotides is sufficient to activate RNP2. In addition, having less than six 5′ nucleotides in a split may compromise hybridization stability (data not shown).

Also in this example, the linker is shown as A₍₁₈₎; however, as with the exemplary 5′ and 3′ AAA_{(N)} blocks, A₍₁₈₎ is exemplary only. The linker that is employed should be a linker that is resistant to trans-cleavage by both the nucleic acid-guided nuclease in both RNP1 and RNP2. Thus, if both RNP1 and RNP2 comprise a DNA-cleaving nucleic acid-guided nuclease, a linker employing ribonucleotides or a chemical linker should be employed. If both RNP1 and RNP2 comprise an RNA-cleaving nucleic acid-guided nuclease, a linker employing deoxyribonucleotides or a chemical linker should be employed. If one of RNP1 or RNP2 comprises a DNA-cleaving nucleic acid-guided nuclease and the other comprises an RNA-cleaving nucleic acid-guided nuclease, a chemical linker should be employed. The length of the linker typically ranges from 15 to 50 nucleotides in length, or the equivalent thereof, or from 20 to 40 nucleotides in length or the equivalent thereof.

FIG. 4 is a simplified diagram showing the sequence of steps in an exemplary cascade assay utilizing blocked split activator molecules and is very similar to the cascade assay utilizing blocked nucleic acids depicted in FIG. 2B. In this embodiment, blocked split activator molecules instead of blocked nucleic acid molecules are used to prevent the activation of RNP2 in the absence of a target nucleic acid of interest. The method (400) in FIG. 4 begins with providing the cascade assay components RNP1 (401), RNP2 (402) and blocked split activator molecules (403). RNP1 (401) comprises a gRNA specific for a target nucleic acid of interest and a nucleic acid-guided nuclease (e.g., Cas 12a or Cas 14 for a DNA target nucleic acid of interest or a Cas 13a for an RNA target nucleic acid of interest—for influenza, a Cas13a for an RNA target of interest to detect an influenza nucleic acid directly, although Cas12a or Cas14 may be used if cDNAs of the sample nucleic acids are prepared) and RNP2 (402) comprises a gRNA specific for an unblocked split activator molecule and a nucleic acid-guided nuclease (again, Cas12a or Cas 14 for a DNA unblocked split activator molecule or a Cas 13a for an RNA unblocked split activator molecule). As described above, the nucleic acid-guided nucleases in RNP1 (401) and RNP2 (402) can be the same or different depending on the type of target nucleic acid of interest and unblocked split activator molecule. What is key, however, is that the nucleic acid-guided nucleases in RNP1 and RNP2 may be activated to have trans-cleavage activity following initiation of cis-cleavage activity.

In a first step, a sample comprising a target nucleic acid of interest (404) (here, a nucleic acid from an influenza virus) is added to the cascade assay reaction mixture. The target nucleic acid of interest (404) combines with and activates RNP1 (405) but does not interact with or activate RNP2 (402). Once activated, RNP1 cuts the target nucleic acid of interest (404) via sequence-specific cis-cleavage, which then activates non-specific trans-cleavage of other nucleic acids present in the reaction mixture, including the blocked split activator molecules (403). At least one of the blocked split activator molecules (403) becomes an unblocked split activator molecule (406) when the blocking moiety (407) is removed. “Blocking moiety” in this context refers to one to eight additional nucleotides on the 5′ or 3′ end—or on both the 5′ and 3′ ends—of the blocked split activator molecules.

Once at least one of the blocked split activator molecules (403) is unblocked, the unblocked split activator molecule (406) can then interact with and activate an RNP2 (408). Because the nucleic acid-guided nucleases in the RNP1s (405) and RNP2s (408) have both cis- and trans-cleavage activity, more blocked split activator molecules (403) become unblocked split activator molecules (406) triggering activation of more RNP2s (408) and more trans-cleavage activity in a cascade. FIG. 4 at bottom depicts the concurrent activation of reporter moieties. Intact reporter moieties (409) comprise a quencher (410) and a fluorophore (411) linked by a nucleic acid sequence. The reporter moieties are also subject to trans-cleavage by activated RNP1 (405) and RNP2 (408). The intact reporter moieties (409) become activated reporter moieties (412) when the quencher (410) is separated from the fluorophore (411), emitting a fluorescent signal (413). Signal strength increases rapidly as more blocked split activator molecules (403) become unblocked split activator molecules (406) triggering cis-cleavage activation of more RNP2s (408) and thus more trans-cleavage activity of the reporter moieties (409).

### Assay Modules

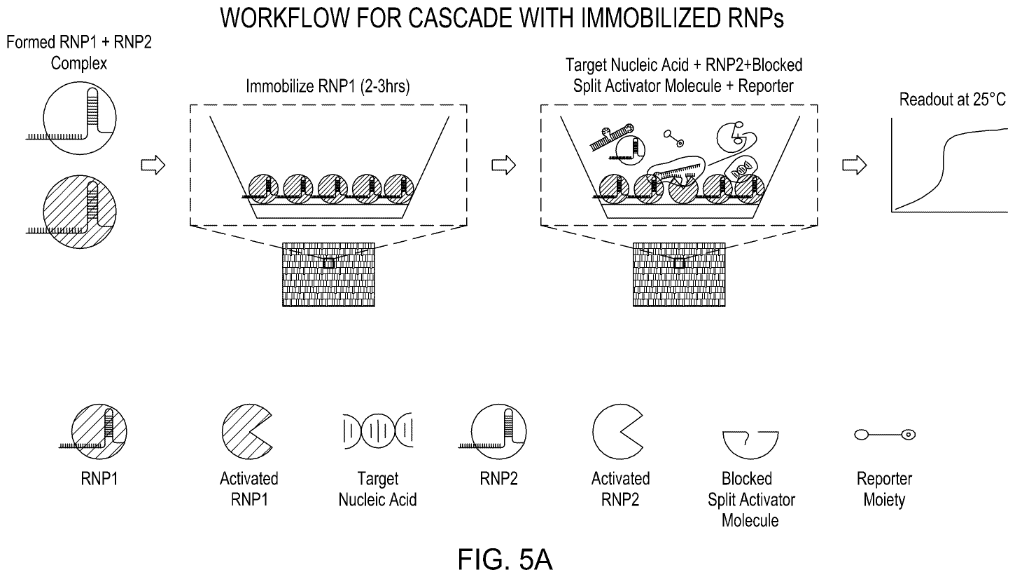

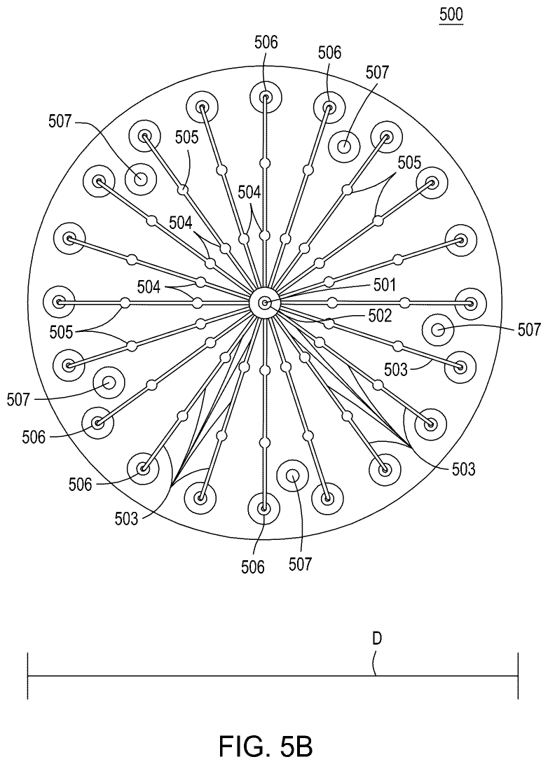

FIG. 5A is a graphic illustration of an exemplary workflow for the sample-splitting and non-sample splitting assay modules where the reactions take place in a partition. As described above, a “partition” may be a well, an isolated region surrounded by interstitial regions or, as described below, a region upon which assay components—specifically RNP1s—are distributed, e.g., on a membrane, microcarriers or beads, or in a packed bed of beads. The workflow in FIG. 5A is essentially identical to that shown in and described in relation to FIG. 4 (i.e., the “blocked split activator molecule” embodiment of the signal boost assay).

At left in FIG. 5A are seen preformed ribonucleoprotein complexes RNP1 and RNP2. Moving right, the RNP1s are distributed (or formed) in a partition (here, only one partition is magnified). Following distribution of the RNP1s into the partitions, the sample, RNP2s, blocked split activator molecules and reporter molecules are distributed into the partition and allowed to contact RNP1. Unactivated RNP1s, a sample comprising an influenza virus nucleic acid, an activated RNP1, an unactivated RNP2, an activated RNP2, a split activator acid molecule, and a reporter moiety are all present. When an influenza virus nucleic acid activates the trans-cleavage activity of RNP1 by binding to RNP1, unblocking of the blocked split activator molecules is initiated where the unblocked split activator molecules bind to RNP2s activating the trans-cleavage activity of the RNP2s thereby initiating more unblocking of blocked split activator molecules and unquenching reporter moieties in a continuing cascade, thereby “boosting” the signal produced by the reporter moieties.

FIG. 5B is an illustration of an exemplary sample splitting assay module (500) similar to that shown in FIG. 1A. Sample splitting assay module (500) comprises an inlet (501), which facilitates fluid displacement via positive or negative pressure; a sample splitting zone (502); twenty fluid channels (503) which connect the sample splitting zone with twenty first reagent wells (504) (i.e., the reagent wells proximal the sample splitting zone); and then connect the first reagent wells (504) with the twenty second reagent wells (505). Also seen are outlets (506), which facilitate fluid displacement (again, via positive or negative pressure) and coupling devices (507), which allow the sample splitting assay modules to be coupled to the pump or plunger or other means for supplying the negative or positive pressure to drive the sample through the fluid channels (503) and/or for coupling the assay module to a sample prep module and/or a detection and imaging module.

The sample, when supplied to the sample splitting zone, may be distributed to the fluid channels (503) passively—that is, the sample is applied to the sample splitting zone and distribution of the sample (i.e., displacement of the sample from the sample splitting zone) into each of the twenty fluid channels (503) is achieved via positive pressure applied to the inlet (501) or negative pressure applied via the twenty outlets (506). The challenge for passive distribution is that the volume of sample distributed to each fluid channel (503) must be consistent, since if the sample aliquot volumes are too different the movement of the sample aliquots through the fluid channels (503) and into the first and second reagent wells (504 and 505) cannot be synchronized. In an alternative, the sample may be distributed to the fluid channels (503) actively, e.g., via, e.g., a twenty-way valve, where the sample aliquots are distributed to each fluid channel (503) one at a time, or e.g., two or four at a time. The volume of the sample aliquot distributed into each fluid channel (503) is controlled by the pressure (or vacuum) driving the sample from the sample splitting zone (502) into each fluid channel and the period of time each valve is open. As an additional alternative, a one-way valve could be used to distribute the sample aliquots to each fluid channel (503) by, e.g., rotating the inlet of each fluid distribution channel (503) to match the valve outlet (not shown).

Note that other configurations for the sample splitting zone are possible, including a member or layer above the sample splitting assay module (500) substrate shown in FIG. 5B, where the sample flows from the outside diameter of the sample splitting assay module (500) toward the middle; that is, where outlets (506) are actually inlets and inlet (501) functions as an outlet such that detection takes place in the more compact middle region of sample splitting assay module (500). In addition, although fluid channels (503) are shown here as being straight, the fluid channels (503) between, e.g., the first (504) and second (505) reagent wells may be serpentine shaped to facilitate mixing between the sample and the RNP1s. Other modalities may be employed as well, such as including features in the fluid channels (503) such as constrictions and/or physical structures such as pillars, creases or fins.

Whether the sample aliquots are delivered to the fluid channels (503) passively or actively, it is important that the sample aliquots be synchronized as they travel through the fluid channels (503) and as they pass through the first (504) and second (505) reagent wells. In addition to employing active sample aliquot distribution, various methods can be used to synchronize the sample aliquots. In one method, the first (504) and second (505) reagent wells could be hydrophilic (that is, coated in a bioinert hydrophilic substance) and the outlets of the first (504) and/or second (505) reagent wells could be hydrophobic (that is, coated in a bioinert hydrophobic substance). In this method, the sample aliquot is drawn in to the first (504) and second (505) reagent wells, but movement of the sample aliquot out of the reagent wells is impeded. The action of drawing the sample aliquot into the reagent wells and impeding flow of the sample aliquot out of the first (504) and/or second (505) reagent wells allows sample aliquots that may be moving more slowly to “catch up” with the sample aliquots that are moving more rapidly.

Another method for synchronizing the movement of the sample aliquots is to constrict the portion of the fluid channels (503) proximate to one or both of the outlets of the first (504) and second (505) reagent wells; that is, to narrow a portion or region of the fluid channels (503) where the fluid channels exit the first and second/or reagent wells (504 and 505). The restriction in flow of the sample aliquots at this (these) “choke points” allows the sample aliquots that may be moving more slowly to “catch up” with the sample aliquots that are moving more rapidly. A third method for synchronizing the movement of the sample aliquots is to employ gravity to increase the elevation of the flow channels (503) as they progress from the sample splitting zone to the first reagent wells (504) and/or to the second reagent wells (505). As the sample aliquots flow up the fluid channels (503), at some point they will be held in place by a force equal to their weight. Yet another method for synchronizing the movement of the sample aliquots is to add membranes to the flow channels (503). The membranes allow the flow of air but significantly slow the flow of fluid. Membranes also assist in reducing air bubbles and foaming that may occur.

Liquid pinning is an exemplary fourth method that may be used for synchronizing the sample aliquots as they flow through the fluid channels (503) encountering the first reagent well (504) and the second reagent well (505). In one embodiment, liquid pinning uses a physical barrier in the fluid channel (503) to slow the movement of the sample aliquot through the fluid channel (503). For example, the diameter of the flow channel (503) can be restricted by a “step” perpendicular to the direction of the flow of the sample aliquot. The sample aliquot must flow over the step, thereby restricting the velocity of the flow. Liquid pinning can be combined, e.g., with using hydrophobic and hydrophilic surfaces to control the velocity of the flow of the sample aliquots, thereby synchronizing the sample aliquots. Indeed, it should be apparent to one of ordinary skill in the art given the present discussion that one, two or more of these methods may be employed for synchronizing the sample aliquots as they travel through the fluid channels (503).

As described in relation to FIG. 1B, the various assay components may be introduced to the sample, (or the sample introduced to the assay components) in different orders. In some aspects, one or more assay components are introduced into the sample before splitting the sample, and thus are aliquoted along with the sample. In other aspects, all assay components (RNP1s, RNP2s, reporter moieties and blocked split activator molecules and, optionally, reagents for target nucleic acid amplification) will all reside in the same partition. In yet other aspects, the RNP1s and the blocked split activator molecules may reside in a first partition and the RNP2s and reporter moieties may reside in a partition “downstream” such that the RNP1s are able to react with the sample and unblock the blocked split activator molecules prior to the unblocked split activator molecules encountering the RNP2s and reporter moieties. In short, many scenarios are possible, the key being that the RNP1s reside in separate, known partitions where they are retained.

The diameter “D” of 2D or parallel module (500) will depend on the volume of the sample to be split and the number of target nucleic acids of interest (i.e., RNP1s) to be tested. The assay modules described herein can be designed and optimized for samples from approximately 10 μL to 1 mL in size.

In an alternative embodiment to the “hub and spoke” configuration of the sample splitting assay module (500), a bifurcation configuration could be employed. That is, instead of taking the sample and distributing the sample into twenty channels all at once or one, two or, e.g., four aliquots at a time, the sample could be halved, then halved again, then halved again and so on until the sample is introduced to a desired number of wells. For example, a 500 μL sample could be split into two 250 μL aliquots, then the two 250 μL aliquots are split into four 125 μL aliquots, then the four 125 μL aliquots into eight 62.5 μL aliquots, then the eight 62.5 μL aliquots into sixteen 31 μL aliquots that are distributed into sixteen wells containing RNP1s and some or all of the other assay components.

Again, the embodiment of the sample splitting assay module (500) described above in this FIG. 5B is exemplary only. As stated above, aside from the sample, other assay components with the exception of the RNP1s may be distributed to or may reside (as one or more lyophilized reagents or soluble reagents) in the sample splitting zone. That is, one or more of the reporter moieties, RNP2s, or blocked split activator molecules may be added to the sample splitting zone. In this embodiment, the sample and the one or more assay components would then be distributed into the fluid channels, which would then flow toward a reagent well comprising RNP1 with no need for a second reagent well. In yet another example, no assay components may be distributed via the sample splitting zone; however, there may be only a single reagent well with all assay components—RNP1, RNP2, blocked split activator molecules and reporter moieties—residing in the single reagent well.

### Uses of the Influenza Virus Nucleic Acid Detection Assay Modules

As described above, the present disclosure describes signal boost assays for detecting an influenza virus nucleic acid in a sample, providing results in less than thirty minutes and allow for multiplexing and minimum workflow yet provide accurate results at low cost. Moreover, the various embodiments of the signal boost assay are notable in that, with the exception of the gRNAs in RNP1, the signal boost assay components may stay the same no matter what target nucleic acid(s) of interest are being detected. As described above, the signal boost assay can be multiplexed for detecting several to many to target nucleic acid molecules simultaneously optionally without amplification of the nucleic acids in the sample. For example, the assay may be designed to detect several to many different known and even unknown influenza virus variants.

The components of the signal boost assay may be provided in various kits for testing at, e.g., point of care facilities, in the field, pandemic testing sites, and the like. In one aspect, the kit for detecting target nucleic acids of interest (i.e., influenza virus nucleic acids) in a sample includes: one or more assay modules, preferably pre-loaded with assay components, where the RNP1s are separated into partitions with, in some embodiments, one or more or all of the additional assay components, including the RNP2s, blocked split activator molecules, and reporter moieties.

Any of the kits described herein may further include a sample collection device, e.g., a syringe, nasal swab, nasopharyngeal swab, or buccal swab for collecting a biological sample from a subject, and/or a sample preparation reagent, e.g., a lysis reagent. Each component of the kit may be in a separate container or two or more components may be in the same container although the RNP1s will be partitioned. In addition, the kit may further include instructions for use and other information.

### EXAMPLES

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Preparation of Nucleic Acids of Interest

Mechanical lysis: Nucleic acids of interest may be isolated by various methods depending on the cell type and source (e.g., tissue, blood, saliva, environmental sample, etc.). Mechanical lysis is a widely-used cell lysis method and may be used to extract nucleic acids from bacterial, yeast, plant and mammalian cells. Cells are disrupted by agitating a cell suspension with “beads” at high speeds (beads for disrupting various types of cells can be sourced from, e.g., OPS Diagnostics (Lebanon NJ, US) and MP Biomedicals (Irvine, CA, USA)). Mechanical lysis via beads begins with harvesting cells in a tissue or liquid, where the cells are first centrifuged and pelleted. The supernatant is removed and replaced with a buffer containing detergents as well as lysozyme and protease. The cell suspension is mixed to promote breakdown of the proteins in the cells and the cell suspension then is combined with small beads (e.g., glass, steel, or ceramic beads) that are mixed (e.g., vortexed) with the cell suspension at high speeds. The beads collide with the cells, breaking open the cell membrane with shear forces. After “bead beating”, the cell suspension is centrifuged to pellet the cellular debris and beads, and the supernatant may be purified via a nucleic acid binding column (such as the MagMAX™ Viral/Pathogen Nucleic Acid Isolation Kit from ThermoFisher (Waltham, MA, USA) and others from Qiagen (Hilden, Germany), TakaraBio (San Jose, CA, USA), and Biocomma (Shenzen, China)) to collect the nucleic acids (see the discussion of solid phase extraction below).

Solid phase extraction (SPE): Another method for capturing nucleic acids is through solid phase extraction. SPE involves a liquid and stationary phase, which selectively separate the target analyte (here, nucleic acids) from the liquid in which the cells are suspended based on specific hydrophobic, polar, and/or ionic properties of the target analyte in the liquid and the stationary solid matrix. Silica binding columns and their derivatives are the most commonly used SPE techniques, having a high binding affinity for DNA under alkaline conditions and increased salt concentration; thus, a highly alkaline and concentrated salt buffer is used. The nucleic acid sample is centrifuged through a column with a highly porous and high surface area silica matrix, where binding occurs via the affinity between negatively charged nucleic acids and positively charged silica material. The nucleic acids bind to the silica matrices, while the other cell components and chemicals pass through the matrix without binding. One or more wash steps typically are performed after the initial sample binding (i.e., the nucleic acids to the matrix), to further purify the bound nucleic acids, removing excess chemicals and cellular components non-specifically bound to the silica matrix. Alternative versions of SPE include reverse SPE and ion exchange SPE, and use of glass particles, cellulose matrices, and magnetic beads.

Thermal lysis: Thermal lysis involves heating a sample of mammalian cells, virions, or bacterial cells at high temperatures thereby damaging the cellular membranes by denaturizing the membrane proteins. Denaturizing the membrane proteins results in the release of intracellular DNA. Cells are generally heated above 90° C., however time and temperature may vary depending on sample volume and sample type. Once lysed, typically one or more downstream methods, such as use of nucleic acid binding columns for solid phase extraction as described above, are required to further purify the nucleic acids.

Physical lysis: Common physical lysis methods include sonication and osmotic shock. Sonication involves creating and rupturing of cavities or bubbles to release shockwaves, thereby disintegrating the cellular membranes of the cells. In the sonication process, cells are added into lysis buffer, often containing phenylmethylsulfonyl fluoride, to inhibit proteases. The cell samples are then placed in a water bath and a sonication wand is placed directly into the sample solution. Sonication typically occurs between 20-50 kHz, causing cavities to be formed throughout the solution as a result of the ultrasonic vibrations; subsequent reduction of pressure then causes the collapse of the cavity or bubble resulting in a large amount of mechanical energy being released in the form of a shockwave that propagates through the solution and disintegrates the cellular membrane. The duration of the sonication pulses and number of pulses performed varies depending on cell type and the downstream application. After sonication, the cell suspension typically is centrifuged to pellet the cellular debris and the supernatant containing the nucleic acids may be further purified by solid phase extraction as described above.

Another form of physical lysis is osmotic shock, which is most typically used with mammalian cells. Osmotic shock involves placing cells in DI/distilled water with no salt added. Because the salt concentration is lower in the solution than in the cells, water is forced into the cell causing the cell to burst, thereby rupturing the cellular membrane. The sample is typically purified and extracted by techniques such as e.g., solid phase extraction or other techniques known to those of skill in the art.

Chemical lysis: Chemical lysis involves rupturing cellular and nuclear membranes by disrupting the hydrophobic-hydrophilic interactions in the membrane bilayers via detergents. Salts and buffers (such as, e.g., Tris-HCl pH8) are used to stabilize pH during extraction, and chelating agents (such as ethylenediaminetetraacetic acid (EDTA)) and inhibitors (e.g., Proteinase K) are also added to preserve the integrity of the nucleic acids and protect against degradation. Often, chemical lysis is used with enzymatic disruption methods (see below) for lysing bacterial cell walls. In addition, detergents are used to lyse and break down cellular membranes by solubilizing the lipids and membrane proteins on the surface of cells. The contents of the cells include, in addition to the desired nucleic acids, inner cellular proteins and cellular debris. Enzymes and other inhibitors are added after lysis to inactivate nucleases that may degrade the nucleic acids. Proteinase K is commonly added after lysis, destroying DNase and RNase enzymes capable of degrading the nucleic acids. After treatment with enzymes, the sample is centrifuged, pelleting cellular debris, while the nucleic acids remain in the solution. The nucleic acids may be further purified as described above.

Another form of chemical lysis is the widely-used procedure of phenol-chloroform extraction. Phenol-chloroform extraction involves the ability for nucleic acids to remain soluble in an aqueous solution in an acidic environment, while the proteins and cellular debris can be pelleted down via centrifugation. Phenol and chloroform ensure a clear separation of the aqueous and organic (debris) phases. For DNA, a pH of 7-8 is used, and for RNA, a more acidic pH of 4.5 is used.

Enzymatic lysis: Enzymatic disruption methods are commonly combined with other lysis methods such as those described above to disrupt cellular walls (bacteria and plants) and membranes. Enzymes such as lysozyme, lysostaphin, zymolase, and protease are often used in combination with other techniques such as physical and chemical lysis. For example, one can use cellulase to disrupt plant cell walls, lysosomes to disrupt bacterial cell walls and zymolase to disrupt yeast cell walls.

### Example II: RNP Formation

Master reaction buffer mixes were prepared with NaCl, MgCl₂, TCEP, MnCl₂, BSA, and other components, as required, with at least 300 μL excess volume in sterile RNase-free tubes. Reaction diluent comprising ROX, reporter and target was prepared and kept at room temperature.

Enzymes were diluted on ice in reaction buffer, as were guide RNAs. RNP1 formations were set up by adding water, buffer, enzyme and gRNAs together, mixing and then heating to 23° C. for 30 minutes. After 30 minutes, the tubes were removed and kept at room temperature until ready to use. RNP1 formations were diluted (on ice) to final reaction concentrations. RNP2 formations were transferred wells in a 96-well plate and kept on ice at least 5 minutes before moving onto the final assembly step.

### Example III: Blocked Nucleic Acid Molecule Formation

Blocked targets (i.e., blocked nucleic acid molecules) containing ribonucleotides were stored dehydrated and required resuspension in buffer at pH 7.5 at 100 μM concentration. Blocked targets comprising only DNA were frozen and thawed for use, then suspended in 1× STE buffer (Sigma, St. Louis MO, US). 25 μL of each target was dispensed into a PCR strip tube and placed into a BioRad (Hercules CA, USA) thermocycler, protocol Hyb0325). After the protocol ended (˜40 minutes), the hybridized blocked targets were stored overnight at room temperature. After ˜16 hours at room temperature, the blocked targets were stored at −20° C. For RNA blocked targets, after hybridization, the hybridized targets were stored at room temperature for 1 hour, then stored at −80° C.

### Example IV: Reporter Moiety Formation

The reporter moieties used in the reactions herein were single-stranded DNA oligonucleotides 5-10 bases in length (e.g., with sequences of TTATT, TTTATTT, ATTAT, ATTTATTTA, AAAAA, or AAAAAAAAA) with a fluorophore and a quencher attached on the 5′ and 3′ ends, respectively. In one example using a Cas12a cascade, the fluorophore was FAM-6, and the quencher was IOWA BLACK® (Integrated DNA Technologies, Coralville, IA). In another example using a Cas13 cascade, the reporter moieties were single stranded RNA oligonucleotides 5-10 bases in length (e.g., r(U)n, r(UUAUU)n, r(A)n).

### Example V: Cascade Assay

Reaction plates were chilled to 4° C. for at least 5 minutes and the reactions were assembled in a place on ice. RNP1, RNP2, targets and blocked targets were added to wells according to experimental design. QuantStudio (Thermo Fisher Scientific, Waltham MA, USA) was set up according to the manufacturer's instructions and was pre-heated to the desired reaction temperature prior to the reaction and the reaction parameters were set and run according to a program.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

I claim:

1. An assay module for identifying one or more influenza virus-related nucleic acids in a sample comprising:
a plurality of partitions comprising reagents to identify the influenza virus-related nucleic acids, wherein the following separate partitions are present:
a partition comprising a reagent to identify an influenza A virus-related nucleic acid;
a partition comprising a reagent to identify an influenza B virus-related nucleic acid;
a partition comprising a reagent to identify an influenza A virus H1 hemagglutinin subtype nucleic acid;
a partition comprising a reagent to identify an influenza A virus H3 hemagglutinin subtype nucleic acid;
a partition comprising a reagent to identify an influenza A virus H5 hemagglutinin subtype nucleic acid;
a partition comprising a reagent to identify an influenza A virus N1 neuraminidase subtype nucleic acid; and
a partition comprising a reagent to identify an influenza A virus N2 neuraminidase subtype nucleic acid.

2. The assay module of claim 1, wherein the reagent to identify the influenza A-related virus nucleic acid identifies an influenza A matrix protein nucleic acid.

3. The assay module of claim 1, wherein the reagent to identify the influenza B-related virus identifies an influenza B hemagglutinin protein nucleic acid and/or nucleoprotein nucleic acid.

4. The assay module of claim 1, further comprising a partition comprising one or more reagents to identify a designated anti-viral mutation in a influenza A virus hemagglutinin subtype nucleic acid or lineage.

5. The assay module of claim 1, further comprising one or more partitions comprising reagents for a control.

6. The assay module of claim 5, wherein the control is a positive control.

7. The assay module of claim 5, wherein the control is a negative control.

8. The assay module of claim 1, further comprising a second partition for a control, wherein the first partition comprises reagents for a positive control and the second partition comprises reagents for a negative control.

9. The assay module of claim 1, further comprising one or more partitions comprising reagents to identify one or more of an influenza A virus H2, H4, H6, H7, H8, H9 or H10 hemagglutinin subtype nucleic acid.

10. The assay module of claim 9, further comprising two or more partitions comprising reagents to identify two or more of an influenza A virus H2, H4, H6, H7, H8, H9 or H10 hemagglutinin subtype nucleic acid.

11. The assay module of claim 10, further comprising three or more partitions comprising reagents to identify three or more of an influenza A virus H2, H4, H6, H7, H8, H9 or H10 hemagglutinin subtype nucleic acid.

12. The assay module of claim 1, further comprising one or more partitions comprising reagents to identify one or more of an influenza A virus N3, N7, N8 or N9 neuraminidase subtype nucleic acid.

13. The assay module of claim 1, further comprising one or more partitions comprising reagents to identify one or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

14. The assay module of claim 13, further comprising one or more partitions comprising reagents to identify two or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

15. The assay module of claim 14, further comprising one or more partitions comprising reagents to identify three or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

16. The assay module of claim 1, further comprising one or more partitions comprising reagents to identify one or more nucleic acids selected from B. parapertussis, B.a pertussis, C. pneumoniae, and/or M. pneumoniae.

17. The assay module of claim 1, wherein the reagents to identify the influenza A virus nucleic acid, influenza B virus nucleic acid, influenza A hemagglutinin subtype nucleic acids and influenza A neuraminidase subtype nucleic acids are ribonucleoprotein complexes.

18. An assay module for identifying one or more influenza virus-related nucleic acids in a sample comprising:
a plurality of partitions comprising reagents to identify the influenza virus-related nucleic acids, wherein the following separate partitions are present:
a partition comprising a reagent to identify an influenza A virus matrix protein nucleic acid;
a partition comprising a reagent to identify an influenza B hemagglutinin protein and/or nucleoprotein nucleic acid;
a partition comprising a reagent to identify an influenza A virus H1 hemagglutinin subtype nucleic acid;
a partition comprising a reagent to identify an influenza A virus H3 hemagglutinin subtype nucleic acid;
a partition comprising a reagent to identify an influenza A virus H5 hemagglutinin subtype nucleic acid;
a partition comprising a reagent to identify an influenza A virus N1 neuraminidase subtype nucleic acid;
a partition comprising a reagent to identify an influenza A virus N2 neuraminidase subtype nucleic acid
a partition comprising a reagent to identify a SARS CoV-2 nucleic acid; and
a partition comprising a reagent for a control.

19. The assay module of claim 19, wherein the control is a positive control.

20. The assay module of claim 19, wherein the control is a negative control.

21. The assay module of claim 19, further comprising a second partition for a control, wherein the first partition comprises reagents for a positive control and the second partition comprises reagents for a negative control.

22. The assay module of claim 19, further comprising one or more partitions comprising reagents to identify one or more of an influenza A virus H2, H4, H6, H7, H8, H9 or H10 hemagglutinin subtype nucleic acid.

23. The assay module of claim 22, further comprising two or more partitions comprising reagents to identify two or more of an influenza A virus H2, H4, H6, H7, H8, H9 or H10 hemagglutinin subtype nucleic acid.

24. The assay module of claim 23, further comprising three or more partitions comprising reagents to identify three or more of an influenza A virus H2, H4, H6, H7, H8, H9 or H10 hemagglutinin subtype nucleic acid.

25. The assay module of claim 19, further comprising one or more partitions comprising reagents to identify one or more of an influenza A virus N3, N7, N8 or N9 neuraminidase subtype nucleic acid.

26. The assay module of claim 19, further comprising one or more partitions comprising reagents to identify one or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

27. The assay module of claim 26, further comprising one or more partitions comprising reagents to identify two or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

28. The assay module of claim 27, further comprising one or more partitions comprising reagents to identify three or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

29. The assay module of claim 28, further comprising one or more partitions comprising reagents to identify four or more nucleic acids selected from seasonal CoVs (OC43, NL63, HKU-1, 229E), SARS CoV-2, RSV, AdV, PIVs 1,2,3,4, HMPV, R/EV nucleic acid, or an emerging influenza virus of concern or specific lineage.

30. The assay module of claim 1, further comprising one or more partitions comprising reagents to identify one or more nucleic acids selected from B. parapertussis, B.a pertussis, C. pneumoniae, and/or M. pneumoniae.
